(19) United States
(12) Patent Application Publication
(10) Pub. No.: US 2018/0099050 A1
(43) Pub. Date: Apr. 12, 2018

(54) DEGRADABLE SILICA NANOSHELLS FOR ULTRASONIC IMAGING/THERAPY

(71) Applicant: The Regents of the University of California, Oakland, CA (US)

(72) Inventors: William C. Trogler, Del Mar, CA (US); Andrew C. Kummel, San Diego, CA (US); Zhe Wu, San Diego, CA (US); Sarah Blair, San Diego, CA (US); Robert F. Mattrey, San Diego, CA (US); Alexander Liberman, San Diego, CA (US); Casey N. Ta, San Diego, CA (US)

(73) Assignee: The Regents of the University of California, Oakland, CA (US)

(21) Appl. No.: 15/706,446

(22) Filed: Sep. 15, 2017

Related U.S. Application Data
(63) Continuation of application No. 14/431,714, filed on Mar. 26, 2015, now abandoned.
(60) Provisional application No. 61/707,794, filed on Sep. 28, 2012.
(60) Provisional application No. 61/845,727, filed on Jul. 12, 2013.

(51) Int. Cl. A61K 41/00; C01B 33/18; A61M 37/00; A61K 9/51; A61K 49/22; B82Y 5/00; B82Y 15/00

(52) U.S. Cl. CPC A61K 41/0033; C01B 33/18; A61M 37/0092; A61K 9/5146; B82Y 15/00; A61K 49/223; Y10T 428/2982; B82Y 5/00; A61K 41/0028

(57) ABSTRACT

Disclosed are methods using degradable silica nanoshells for local intra-operative ultrasound marking; tumor detection via systemic injection; and nanoshell enhanced ultrasonic ablation of tumors.

### CROSS REFERENCE TO RELATED APPLICATIONS

This patent document is continuation of U.S. application Ser. No. 14/431,714, filed Mar. 26, 2015, which is a 35 U.S.C. § 371 National Stage application of PCT Application No. PCT/US2013/062436, filed Sep. 27, 2013, which claims priority under 35 U.S.C. § 119 from U.S. Provisional Application Ser. No. 61/707,794, filed Sep. 28, 2012, and U.S. Provisional Application Ser. No. 61/845,727, filed Jul. 12, 2013, the disclosures of which are incorporated herein by reference.

### STATEMENT REGARDING FEDERALLY SPONSORED RESEARCH OR DEVELOPMENT

This invention was made with government support under Grant No. CA151140 awarded by the National Institutes of Health. The government has certain right in the invention.

### TECHNICAL FIELD

The disclosure relates to nanostructures and methods of making and using the same. More particularly, the disclosure provides hollow nanoshells useful for drug delivery, imaging, gene transfer, cancer treatment and sensing.

### BACKGROUND

The current standard ultrasound technology for screening can reliably detect tumors 5-10 mm in size depending on the tumor. Earlier detection of tumors will allow surgeons to resect smaller volumes of tissue and make patients more likely to be recurrence free. Surgical resection remains the most effective treatment for most solid organ cancers like breast cancer in order to prevent recurrence, progression, and ultimately spread of disease; therefore, there is an increased need for small tumors to be precisely identified and localized.

For breast cancer, mammographic screening has been shown to decrease mortality rates by 15-25% in several large randomized prospective studies. This data suggests that further improvement in screening accuracy could increase survival. Mammographic sensitivity is impaired for non-calcified masses in radiographically dense breast tissue. In North America, breast ultrasound is most often a targeted examination, limited to the area of concern based on palpation or mammography. MRI has been extremely beneficial for screening high-risk women, evaluating silicone implants, and possibly monitoring response to neoadjuvant chemotherapy. MRI is starting to be employed preoperatively to determine a patient's eligibility for breast conservation therapy (BCT). Unfortunately there has not been a demonstrated benefit to pre-operative MRI for improving outcomes of breast conservation. Furthermore, pre-operative MRI is an expensive procedure with a relatively high false positive rate leading to more biopsies and increasing rates of mastectomy. Currently, whole breast ultrasound screening can reliably detect ˜10 mm diameter tumors. For lesions that remain equivocal after mammographic and standard sonographic (Ultrasound (US)) evaluation, it has been postulated that contrast enhanced sonography (CEUS) could be the problem-solving method.

High Intensity Focused Ultrasound (HIFU) is employed to locally heat and ablate tumors via a local increase in temperature. HIFU is a minimally invasive therapy which compared to most radiation techniques minimizes damage outside tumors and is extremely low cost. Frequently MRI is employed for guidance and to monitor the temperature of the tumor during HIFU. HIFU is also employed for ultrasound assisted local drug delivery; for example HIFU can be employed to break drug carrying liposomes in the vicinity of a tumor. Currently, in the USA, HIFU is approved to treat uterine fibroids, however; internationally it is also employed to treat many types of cancer. HIFU therapy has the advantage over other types of radiation because the ultrasound energy has no cumulative effect on the tissue between the tumor and transducer thereby allowing many treatments of the same tumor. This is particularly valuable for controlling difficult cancers that are metastatic and persistent, such as prostate cancer. Contrast enhance ultrasound (CEUS) is used in conjunction with HIFU to lower the power (mechanical index) during HIFU. Conventional HIFU-CEUS requires continuous administration of CEUS agents (conventional microbubbles) since the CEUS agents are not retained well in tumors and the HIFU treatments are time intensive.

### SUMMARY

The reported positive margin rate from wire localized excisions of breast cancers is approximately 20-50% thereby requiring second surgeries (re-excision); however, by preoperatively injecting a radioactive seed into the tumor under CT guidance, the re-excision rate is halved because the surgeon can constantly reorient the dissection to place the seed in the center of the specimen. Unfortunately, radioactive seed localization has several safety challenges, only single foci can be localized, and incisions are required to implant the seeds, so it is rarely employed. As a safe alternative, the disclosure provides gas-filled hollow Fe-doped silica particles, which can be used for ultrasound-guided surgery even for multiple foci. The function of the Fe doping is to render the silica shells biodegradable. The particles are synthesized through a sol-gel method on a polystyrene template, and subsequently calcinated to create hollow, rigid micro/nanoshells. The Fe-doped silica shell is derived from tetramethyl orthosilicate (TMOS) and iron (III) ethoxide, which forms a rigid, mesoporous shell upon calcination. The micro/nanoshells are filled with perfluoropentane (PFP) vapor or liquid. The fluorous phase is contained within the porous shell due to its extremely low solubility in water. Considerable testing of particle functionality, signal persistence and acoustical properties have been performed in various phantoms including ultrasound gel, chicken breast, and excised human mastectomy tissue. In vitro studies have shown that continuous particle imaging time is up to approximately 45 minutes, and will persist for over five days. Furthermore, in vivo particle injection longevity studies have been performed in a mouse tumor model which is consistent with in vitro data showing signal presence even ten days post injection. These silica spheres may also be used a sensitizing agent in high intensity focused ultrasound (HIFU). Traditional ultrasound imaging agents are based on soft shell (e.g., albumin) encased gas bubbles and pose several potential drawbacks such as poor in vivo persistence (minutes) and high risk (cardiac complications) during continuous perfusion. Preliminary in vitro results in HIFU ablation in an agar tissue phantom model suggest that very few particles are needed in order to develop a sensitizing effect to HIFU (approx. 1-10 μg/ml particles/agar varying by particle size). The disclosure also provides a technique to fill the particles with perfluorocarbon liquid which vaporizes upon exposure to HIFU thereby further increasing the sensitivity compared to gas filled particles.

The disclosure provides methods and compositions for use comprising (1) the pure silica and biodegradable iron doped silica nanoshells can be used to find tumors via IV injection. Silica shells tend to accumulate in late state tumors such that a single bolus injection can be employed to detect tumors. The existing technique relies on an injection of soft particles for contrast enhanced ultrasound to enable the kinetics of the blood flow to be employed to image tumors. The silica shells instead are just retained by the tumor so their mere presence is employed to show the existence of a tumor. (2) Nanoshell Enhanced Ultrasonic Ablation 1—High intensity focused ultrasound (HIFU) is currently employed in the USA to treat fibroids (30% of all women post menopause have fibroids). In Canada and the UK, it is also used to treat prostate cancer, liver cancer, and some other solid tumors. Conventional ultrasound contrast agents are often employed to allow lower power for faster treatment and to avoid un-desired or off target thermal damage. HIFU without contrast agents works by raising the tissue temperature in the tumor to 50-90° C. With contrast agents, at least two additional effects occur to improve the HIFU therapy. Firstly, the contrast agent attenuates ultrasound to increase the local heat deposited in the region of the contrast agent. Secondly, HIFU also attacks the tumor because the cavitating contrast agents mechanically damage the tissue, including the vasculature. Conventional contrast agents for HIFU require continuous infusion, because they are not retained by the tissues and have a short circulation time. The silica nanoshells remain in circulation for several hours, and are selectively retained by late stage tumors. This enables shorter treatment times as well as a single injection, which carries much lower risk. (3) Nanoshell Enhanced Ultrasonic Ablation 2—Experiments show that 500 nm nanoshells can be filled with perfluorocarbon liquids. This liquid filling enables a new application in which high power ultrasound converts the nanoshells to 1 mm gas bubbles via coalescence. This enables the particles to be used to occlude the vascular supply of a tumor. Data have been obtained showing that the liquid filled 500 nm nanoshells can be converted to many 1 mm gas bubbles using high power ultrasound. (4) Imaging Mode—the disclosure shows that the particles have high contrast for even 50 microliter injections in tissue when imaged with color Doppler ultrasound.

In another embodiment, the compositions and methods of the disclosure include combining nanoshell enhanced ultrasonic ablation (HIFU) with administration of viral therapy/liposomal or polymeric formulations/chemotherapeutic agents. In this embodiment administration may be (a) local: the therapeutic is delivered to the cavity of tissue liquefied by mechanical ablation induced by cavitation of the nanoshell compositions of the disclosure following cavitation and rupture from interaction with ultrasound; or (b) systemic: the inflammatory response which follows ablative therapy may enhance either oncolytic viruses, antibody therapy, or chemotherapuetic drug delivery and retention specifically in the site of focal ablation.

In another embodiment, nanoshell and HIFU “tissue drilling” is performed by local injection of perfluorocarbon liquid or gas filled nanoshells of the disclosure wherein application of HIFU leads to mechanical cavitation of the nanoshels that liquefy the tissue at the injection site. The liquefied tissue can be removed with vacuum to create a cavity which can be refilled repeatedly with additional nanoshells for further HIFU application to enlarge or deepen the cavity for rapid ablation of large tissue volumes.

In yet another embodiment, longterm imaging markers are provided comprising perflurocarbon (PFC) liquid filled nanoshells, functionalized with a fluorinated trialkoxysilane for extremely long term in vivo ultrasound imaging. The fluorinated trialkoxysilane makes the particles “non-wetting.” This will prevent PFC from escaping the particles and also particle degradation.

The compositions and method of the disclosure can be used to treat benign prostatic hyperplasia; combinatorial treatment of liver cancer; liquification of uterine fibroids; liquification of breast fibroadenomas; treatment of prostate cancer; non-surgical treatment of breast cancer; combinatorial treatment of head and neck cancers; long-term markers for breast/prostate cancer and other disease and methods where tissue ablation is useful.

The disclosure provides a method for producing perfluorocarbon liquid filled nanoshell. In one embodiment, the perfluorcarbon filled nanoshell is functionalized with various alkoxysilanes for improved ultrasound response.

The disclosure also provides a method of imaging a cancer comprising administering the nanoshells of the disclosure to a subject, and imaging the subject to identify localization of the nanoshells

The disclosure provides a method of treating a hyperplasia comprising administering nanoshells of the disclosure to the hyperplasia tissue, contacting the nanoshells with sufficient energy to cause disruption and cavitation and sufficient to liquefy hyperplasia tissue near the nanoshells.

The disclosure provides a method of treating a solid tumor and various cancers (e.g., liver cancer) comprising administering nanoshells of the disclosure to the cancer tissue, applying HIFU to the nanoshells with sufficient energy to cause disruption and cavitation and sufficient to liquefy the cancer tissue near the nanoshell, optionally removing the liquefied tissue and delivery of a therapeutic agent to the site of liquefied tissue.

The disclosure also provides a method of treating a uterine fibroids comprising administering nanoshells of the disclosure to the uterine fibroid tissue, contacting the nanoshells with sufficient energy to cause disruption and cavitation and sufficient to liquefy uterine fibroid tissue near the nanoshells.

The disclosure also provides a method of treating a breast cancer or breast fibroadenomas comprising administering nanoshells of the disclosure to the breast tissue, applying HIFU to the nanoshells with sufficient energy to cause disruption and cavitation and sufficient to liquefy the cancer or fibroadenoma tissue near the nanoshells.

The disclosure also provides a method of treating a prostate cancer comprising administering nanoshells of the disclosure to the breast tissue, applying HIFU to the nanoshells with sufficient energy to cause disruption and cavitation and sufficient to liquefy the cancer tissue near the nanoshells.

The disclosure also provides a method of treating head and neck cancers comprising administering nanoshells of the disclosure to the cancer tissue applying HIFU to the nanoshells with sufficient energy to cause disruption and cavitation and sufficient to liquefy the cancer tissue near the nanoshells.

The disclosure also provides a method of treating renal cell carcinomas comprising administering nanoshells of the disclosure to the cancer tissue applying HIFU to the nanoshells with sufficient energy to cause disruption and cavitation and sufficient to liquefy the cancer tissue near the nanoshells.

In any of the foregoing embodiments, the liquefied tissue can be removed. In a further embodiment, the cavity where the liquefied tissue is or was can be injected with a therapeutic agent. In yet a further embodiment, the therapeutic agent comprises a chemotherapeutic, a liposomal formulation, an oncolytic virus, an antibody, a protein, a polypeptide, a small molecule agent or any combination thereof.

The disclosure provides a nanostructure comprising a degradable nanotemplate comprising a polyamine or polycarboxylic acid functionalized surface layer; and a layer of a compound comprising the structure of Formula I:

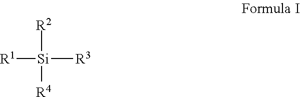

wherein, R¹-R⁴ are independently selected from the group consisting of H, D, optionally substituted (C₁-C₁₈)alkyl, optionally substituted (C₁-C₁₈)alkenyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether; wherein at least one of R¹-R⁴ is an optionally substituted alkoxy, and wherein if three of R¹-R⁴ are methoxy groups then the fourth R group is selected from the group consisting of D, optionally substituted (C₁-C₁₈)alkyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted (C₂-C₁₈)alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether. In another embodiment, wherein at least two of R¹-R⁴ are optionally substituted alkoxy groups. In yet another embodiment, at least three of R¹-R⁴ are optionally substituted alkoxy groups. In another embodiment, R¹-R³ are optionally substituted alkoxy groups, and R⁴ is an optionally substituted (C₂-C₁₈)alkoxy group. In yet another embodiment of any of the foregoing the degradable nanotemplate comprises a polyamine functionalized surface layer, wherein the polyamine is a homopolymer of amino acids or an aliphatic amine with primary amine groups on the polymer backbone or wherein the nanotemplate comprises a cationic polymer or molecular anchor with a cationic headgroup. In another embodiment, the polyamine is selected from the group consisting of poly-L-lysine, poly-L-arginine and polyornithine. In another embodiment, the aliphatic amine is polyethyleneimine. In yet another embodiment, the degradable nanotemplate comprises a polyamine or polycarboxylic acid functionalized polystyrene or latex surface layer. In yet another embodiment of any of the foregoing, the nanotemplate is from 10 nm to 3000 nm in size. In yet another embodiment of any of the foregoing the layer further comprises iron (III) ethoxide. In a further embodiment, the nanostructure is calcinated at an elevated temperature to degrade the nanotemplate so as to afford a hollow silica nanostructure or hollow silica-iron nanostructure. In another embodiment, the nanostructure is treated with an organic solvent to dissolve the nanotemplate so as to afford a hollow silica nanostructure or hollow silica-iron nanostructure. In yet another embodiment, of any of the foregoing the nanostructure is a hollow nanoshell. In a further embodiment, the hollow nanoshell has a diameter between 10 nm to 3000 nm. In yet a further embodiment, a perhalocarbon is introduced into the nanoshell. In yet a further embodiment, the perhalocarbon is a perfluorocabon (PFC) liquid or gas.

The disclosure also provides a method for imaging neoplasms in a subject, comprising, administering the nanostructure of any preceding embodiments to the subject, and imaging neoplasms by detecting the nanostructure via ultrasound.

The disclosure also provides a method for treating neoplasia in a subject, comprising, administering the nanostructure of any of the foregoing embodients to the subject, heating the nanostructures located in neoplasms by using high intensity focused ultrasound (HIFU) so as to damage the neoplasms. In a further embodiment, the method includes coalescing the perfluorocarbon liquid in the nanostructures in neoplasms to form gas bubbles via HIFU. In yet a further embodiment, the neoplasms are malignant neoplasms.

The disclosure also provide a method to produce a nanostructure, including nanoshells, of the disclosure comprising mixing polystyrene or latex beads with a polyamine, polyamino acids, cationic polymers, or molecular anchors with a cationic headgroup in a solution to form a degradable nanotemplate; adding a mono-, di-, tri- or teta-aalkoxysilane to the aqueous solution so that the alkoxysilane is deposited as a layer onto the surface of the degradable nanotemplate. In one embodiment, the ratio of the polyamine to polystyrene beads is from 1:1 to 10:1 v/v. In another embodiment, iron (III) ethoxide/trimethyl borate is added to the solution. In yet a further embodiment of any of the foregoing, the method further comprises isolating the nanostructure from the aqueous solution by using centrifugation; washing the nanostructure by using an alcohol based solvent; collecting the nanostructure via centrifugation; and drying the nanostructure under vacuum. In another embodiment, the process further comprises calcinating the nanostructure to obtain a hollow silica nanostructure or hollow silica-iron nanostructure.

The disclosure also provide a hollow silica nanostructure of any of the foregoing embodiments or developed by any of the foregoing methods. In one embodiment, the hollow silica nanoshell is porous. In another embodiment, the nanoshell has pores of about 1 nm to about 100 nm. In yet a further embodiment, the nanoshell has a surface area of at least 100 m²/gram to 1000 m²/gram (e.g., about 400 m²/gram). In yet still another embodiment, the nanoshell is more fragile compared to a nanoshell made using a tetra- or unsubsituted trialkoxysilane. In another embodiment, the nanoshell is a perfluorocarbon liquid or gas filled nanoshell. In yet another embodiment, the hollow silica nanoshell (HSN) can be activated by HIFU for B-mode and contrast enhanced ultrasounds in both intravenous or direct delivery of hollow silica nanoshell to the target tissue. In yet another embodiment, the presence of hollow silica nanoshells can be detected by activation of HIFU for directed imaging and ablative therapy.

The disclosure also provides a method of imaging a cancer or tumor, comprising administering to a subject having the cancer or tumor a hollow silica nanoshell (HSN) doped or undoped with iron as described in the foregoing embodiments; and ultrasonic imaging the subject, wherein the HSN emit a detectable signal and wherein the HSN concentrates at the tumor or cancer site. In one embodiment, the HSNs are filled with perfluorocarbon gas or liquid. In another embodiment, the HSNs are iron doped. In yet another embodiment, the HSNs are about 10 to 3000 nm in diameter.

The disclosure also provides a method of treating a cancer or tumor, comprising administering to a subject having the cancer or tumor a hollow silica nanoshell (HSN) doped or undoped with iron as described in the foregoing embodiments; and contacting the HSN with a frequency that causes the HSN to generate heat at the site of tumor or cancer thereby killing the tumor or cancer cells. In one embodiment, the method further comprises contacting the HSN with a frequency that causes cavitation and rupture of the HSN. In yet another embodiment, the HSN are filled with perfluorocarbon gas or liquid.

The disclosure also provides a method of treating a hyperplasia comprising administering a hollow silica nanoshell (HSN) doped or undoped with iron as described in the foregoing embodiments to the hyperplasia tissue, contacting the nanoshells with sufficient energy to cause disruption and cavitation and sufficient to liquefy hyperplasia tissue near the nanoshells.

The disclosure also provides a method of treating a liver cancer comprising administering a hollow silica nanoshell (HSN) doped or undoped with iron as described in the foregoing embodiments to solid tumor tissue, contacting the nanoshells with sufficient energy to cause disruption and cavitation and sufficient to liquefy the cancer tissue near the nanoshell, optionally removing the liquefied tissue and delivery a therapeutic agent to the site of liquefied tissue. In yet another embodiment, the therapeutic agent comprises a chemotherapeutic, a liposomal formulation, an oncolytic virus or any combination thereof.

The disclosure also provides a method of treating a uterine fibroids comprising administering a hollow silica nanoshell (HSN) doped or undoped with iron as described in the foregoing embodiments to the uterine fibroid tissue, contacting the nanoshells with sufficient energy to cause disruption and cavitation and sufficient to liquefy uterine fibroid tissue near the nanoshells.

The disclosure also provides a a method of treating a breast cancer or breast fibroadenomas comprising administering a hollow silica nanoshell (HSN) doped or undoped with iron as described in the foregoing embodiments to the breast tissue, contacting the nanoshells with sufficient energy to cause disruption and cavitation and sufficient to liquefy the cancer or fibroadenoma tissue near the nanoshells.

The disclosure also provides a method of treating a prostate cancer comprising administering a hollow silica nanoshell (HSN) doped or undoped with iron as described in the foregoing embodiments to the breast tissue, contacting the nanoshells with sufficient energy to cause disruption and cavitation and sufficient to liquefy the cancer tissue near the nanoshells.

The method of treating head and neck cancers comprising administering a hollow silica nanoshell (HSN) doped or undoped with iron as described in the foregoing embodiments to the cancer tissue contacting the nanoshells with sufficient energy to cause disruption and cavitation and sufficient to liquefy the cancer tissue near the nanoshells.

The disclosure also provides a method of treating renal cell carcinomas comprising administering a hollow silica nanoshell (HSN) doped or undoped with iron as described in the foregoing embodiments to the cancer tissue contacting the nanoshells with sufficient energy to cause disruption and cavitation and sufficient to liquefy the cancer tissue near the nanoshells.

In any of the foregoing embodiment and methods of treatment, the liquefied tissue is removed. In a further embodiment, the cavity where the liquefied tissue was is injected with a therapeutic agent. In yet a further embodiment, the therapeutic agent comprises a chemotherapeutic, a liposomal formulation, an oncolytic virus or any combination thereof.

### DESCRIPTION OF DRAWINGS

FIGS. 1A-1D shows electron microscopy images of hollow silica particles. (A) Transmission electron microscopy image of 500 nm iron doped silica nanoshells. (B) Scanning electron microscopy of 500 nm Iron doped Silica Nanoshells. (C) (Left) Scanning electron microscopy (SEM) image of calcined hollow boron-doped 2 micron silica nanoparticles. (1 micron scale bar). (Right)—Transmission electron (TEM) microscopy image of 100 nm hollow silica nanoparticles (the scale bar is 100 nm). Note the thin wall (10 nm for pure silica nanoshells or about 40 nm for iron-doped nanoshells) and uniform size. For ultrasound imaging, the particles are filled with gas. For targeting, the silica shell is functionalized. (D) Scheme 1 depicts Synthesis of SiO₂ Nanoshells. Scheme 2 depicts Synthesis of Fe-doped, Silica Nanoshells.

FIGS. 2A-B shows microshell Imaging In-Vivo. (A) The in vivo silica shell signals as a heat map overlay within an ovarian cancer (red arrow) in a mouse model. Most of the signal is from the vasculature at the edge of the tumor. (B) The cross sections of an artery (green arrow) and a vein (blue arrow) are clearly marked by the nano-shells with high resolution. The results are consistent with 1 mm resolution.

FIG. 3A shows persistence in an In vivo Model. 50 μl of control microbubbles, 2 μm shells and 500 nm shells were injected into New Zealand White Rabbit thighs and imaged over the course of four days. Shown in the left columns are the control microbubbles; 50 μl were injected containing 108 microbubbles/ml. All injections were imaged at a MI of 1.9 at 7 MHz with color Doppler using the Siemens Sequoia. Day 0 corresponds to imaging within 15 min of the injection. Note that signal persisted for 4 days when either formulation of silica particles were injected. Microbubbles given as 10⁸ (left column).

FIG. 3B shows in vivo comparison of injection volumes. Nu/Nu mice seeded with PyVmT tumor cells were grown to ˜1000 mm3 and then injected with 500 nm Fe-doped SiO2 nanoshells. Two different injection volumes, 50 μl and 100 μl, were tested at a nanoshell concentration of 4 mg/ml. Animals were imaged after the initial injection and then 1, 24, or 72 hours post injection. Note, each image comes from a different animal indicating very high animal-to-animal and injection-to-injection consistency.

FIGS. 4A-4B shows ex vivo injection into excised mastectomy tissue. (A) Ultrasound color doppler image from a 100 μl injection of gas filled Fe-doped silica 500 nm FITC functionalized nanospheres into mastectomy tissue along the tumor margin. Note: asymmetric contrast is due to shadowing. (B) Fluorescent microscopy scan at 5× magnification from a cross-sectional cut of the injection site seen in image (A). Green area is attributed to fluorescence from FITC conjugated onto the surface of the nanospheres.

FIG. 4C shows color doppler imaging (CDI) of stationary gas filled microshells in human breast tumor tissue. 100 μL of a 2 mg/ml suspension 2 micron silica particle dyed with 1 drop of India ink were injected. (left) CDI of the ˜4×10¹⁰ microshells. The bright regions are 5 mm×5 mm×5 mm≈100 μM³=100 μL. Two injections clearly outline a known tumor. (right) Photograph of injection region showing the India ink dye.

FIG. 5A-5D shows testing of gas-filled microshells in a mouse. (A) Dissected Nu/Nu mouse with an intraperitoneal IGROV-1 Ovarian tumor (see red arrow: white mass on right side of image). 200 μg of PFP filled 2 μm particles were diluted into 3 ml of saline and injected into the peritoneum and then perfused into the blood. (B) CPS imaging of the particles through a cross section of the tumor 1 hour after quasi IV injection. (C) B-mode image through a cross section of the tumor 1 hour after quasi IV injection. (D) Overlay image using several frames from CPS imaging and B-mode to show an integrated heat map of signal from the particles. For all the images, the red arrow points to the tumor, the green arrow points to the spinal column and the blue arrow points to the bottom of the mouse.

FIG. 6 shows confocal results of cell endocytosis of folate functionalized silica shells. (Left) After functioning with Folate targeting ligand, the nano silica shells (green) are readily endocytosed by Hela cells. Note the nanoshells (green) are inside the membranes. (Right) Selectivity of Folate Targeting Silica NS. Folate targeted NPs (green) show higher preference for folate receptor rich HeLa Cancer Cells (red) compared to HFF-1 Normal Cells.

FIGS. 7A-7B shows CPS imaging of liquid filled 2 μm SiO₂ shells (200 ug/ml) before and after large bubble stimulation. (A) At an MI of 0.97 liquid particles behave and appear the same as traditional CEUS bubbles. (B) At an MI of 1.9 large bubble stimulation is observed.

FIGS. 8A-8E shows a biodistribution study with healthy Nu/Nu mice. Mice were injected via the tail vein with In-111 labeled with 100 μl (4 mg/ml) of gas filled 500 nm Fe-doped SiO₂ nanoshells and then imaged by gamma scintigraphy. A) Imaging at 0 hours-during the injection. B) Imaging 1 hour post injection. C) Imaging 24 hours post injection D) Imaging 72 hours post injection. E) Gamma counter readings of harvested organs normalized by mass of the individual organs.

FIGS. 9A-9B shows in vivo nanoshell enhanced ultrasonic ablation. (A) A thermal lesion is produced by highly energetic ultrasonic ablation without nanoshell enhancement after 60 seconds of exposure. (B) Both mechanical and thermal damage are produced with nanoshell enhancement after only 30 seconds of ultrasonic ablation at an equivalent power.

FIGS. 10A-10D shows ex vivo HIFU of excised mastectomy tissue. 50 ul at 4 mg/ml of PFC liquid filled 500 nm nanoshells were injected intratumorally ex vivo. (A) Color Doppler ultrasound imaging displays the location of the nanoshells allowing for better targeting of the HIFU transducer. (B) B-mode image of tissue prior to HIFU. (C) HIFU is applied for 1 min at 1.1 MHz and 3 MPa with a 2% duty cycle. Bubble cavitation/formation is readily observed. (D) After HIFU a pocket (black spot) filled is created which is filled with the liquefied tissue.

FIGS. 11A-11C shows nanoshell enhanced HIFU in vivo in Py8119 Tumor Bearing Nu/Nu Mice. 800 ug of 500 nm liquid PFP filled nanoshells were administered IV. HIFU was applied 24 hours after administration for 1 min at 3 MPa and 1.1 MHz with a 2% duty cycle. A) Before HIFU B) During HIFU, bubble movement/generation can be noticed at the focal zone. C) Post HIFU. Blackened area at HIFU focus is liquefied tissue.

FIGS. 12A-12G shows intratumoral nanoshell ultrasound imaging longevity. 50 μl of 500 nm PFP gas filled Fe—SiO2 nanoshells at a concentration of 4 mg/ml were injected intratumorally into eight Py8119 tumor bearing mice and imaged by color Doppler imaging. The mechanical index was 1.9 with an imaging frequency of 7 MHz. (A) Imaging immediately after injection. (B) Imaging at 1 day post injection (C) Imaging at 3 days post injection (D) Imaging at 5 days post injection (E) Imaging at 7 days post injection (F) imaging at 10 days post injection. (G) Color Doppler signal width was measured and plotted vs time post injection. Error bars signify standard deviations.

FIG. 13 depicts a scheme for pegylation of silica shells.

### DETAILED DESCRIPTION

As used herein and in the appended claims, the singular forms “a,” “and,” and “the” include plural referents unless the context clearly dictates otherwise. Thus, for example, reference to “a nanoshell” includes a plurality of such nanoshells and reference to “the cell” includes reference to one or more cells and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Microbubble based contrast agents are clinically used to enhance the ultrasound (US) echo signals. Commercially manufactured US contrast agents have lipid, polymer, or protein shells encapsulating either air (Albunex, Levovist, Sonovist) or perfluorocarbon gas (Definity, Optison). These microbubbles generate significant contrast at relatively low acoustic pressure with color Doppler, power Doppler, or contrast specific imaging techniques available on commercial systems. Ultrasonic (US) pulses, particularly at microbubble resonance induce microbubble destruction at mechanical indices (MI) below 0.3; MI is a measure of US energy that is the ratio of peak negative pressure and the square root of the transmit frequency. Microbubble destruction causes a de-correlation between two consecutive US pulse that is visible as color on Doppler imaging that has been termed stimulated acoustic emission (SAE). While microbubble destruction can also be detected with contrast specific imaging methods, these techniques were developed to detect the non-linear behavior of microbubbles when exposed to non-destructive US pressures at very low MI. Because tissues respond linearly to US while the microbubbles respond non-linearly, these techniques are extremely sensitive to the presence of microbubbles and can detect a single microbubble. The non-linear response of microbubbles is related to their ability to expand and contract when exposed to US, which is controlled by the elasticity of the encapsulating shell. Tiemann et al. demonstrated that using SAE of air filled cyanoacrylate microbubbles, a high signal is obtained using Doppler imaging from a stationary bolus of particles which have been cast into gelatin. However, the continuous imaging time is brief and the particles are not entirely uniform in size. Typically, ultrasound contrast agents are administered intravenously to study vasculature; due to the typical size of the microbubbles (1-5 μm), they cannot escape the vasculature. Consequently, ultrasound contrast agents have only been employed in the detection and diagnosis of tumors by studying aberrant tumor vasculature due to angiogenesis.

Silica particles have been explored recently as ultrasound contrast agents. Lin et al. tested hollow silica capsules with CPS at high MI in a liquid filled plastic beaker. Hu et al. developed hollow silica microspheres that were imageable at low MI (0.06) and injected them into male rat spermary and imaged with CEUS. Wang et al. were able to effectively encapsulate pefluorohexane liquid into mesoporous silica nanoshells and then perform thermal ablative HIFU in vivo. Silica shells or any other hard shell CEUS have not been used by other groups to find tumors.

Hollow silica nanoshells are potentially applicable to drug delivery and imaging. Hollow silica nanoshells have uniform and stable wall structures with excellent long term stability. Their size can be controlled by using polymer templates for their formation with well-defined diameters accessible from emulsion polymerization used to form the polymer templates. The porosity of the silica shell is convenient for loading and releasing of gases, drugs or used to contain a heavy element (e.g. metal nanoparticle) or magnetic oxides for X-ray or magnetic contrast reagents. The surface of the hollow silica shell is easily functionalized by grafting biofunctional groups that may combine with targeting proteins, antibodies, cells, or tissues. Furthermore, the disclosure demonstrates that the rigidity/fragility of the shell can be selectively prepared for a particular use, frequency of US and the like.

Many methods have been employed to fabricate hollow silica spheres, such as colloidal templating and layer-by-layer (LbL) self-assembly techniques. Colloidal particles were used to make core-shell nanospheres of gold, silver, CdS, ZnS and polymer beads; however, the inorganic templates are difficult to remove from the core-shell spheres. For those hollow spheres templated with polymers, their size and uniformity depend on the species and density of the surface functional groups, which makes size control difficult. The basis of the LbL technique is the electrostatic attraction between the charged species deposited. But this method involves numerous synthetic steps which make large scale production impractical. The challenge of hollow silica nanoparticle technology is to find a convenient and inexpensive method to fabricate hollow silica nanoshells with uniform, stable shell walls, and at the same time this shell should have acceptable porosity and a narrow size distribution.

There is, currently, no scalable inexpensive method for making uniform size distributions of hollow nanoshells. Current nanoparticles used for drug delivery and sensing are solid. Hollow nanoshells offer the possibility of filling with a payload of drug, imaging agent, or other material. The outer and inner surfaces could also be differentially functionalized.

During the past decade, there has been intense interest about the fabrication of hollow silica nanoparticles because of their applications such as drug delivery, ultrasound imaging, catalyst, filters, photonic band gap materials. In reported fabrication protocols, colloidal templating and layer-by-layer (LbL) self-assembly technique are most usually used. Colloidal templates used include gold, silver, CdS, ZnS and polymer beads. Polystyrene (PS) beads are attractive nanoscale templates since they are inexpensive and their size is easily varied. Furthermore their surface can be functionalized by chemical and physical techniques. Finally they are well-suited to make hollow particles since the polystyrene template can easily be removed by calcination or dissolution. Calcination can remove the latex cores and give the hollow SiO₂ nanoparticles. For example, the size and the uniformity of the nanoparticles depend in-part upon the density of the surface functional groups which makes the size control difficult.

Poly-L-lysine (PL) is one of the simplest polyamino acids with a pH-dependent structure and has been applied in many syntheses of ordered silica structure.

This disclosure provides a method of synthesis of hollow silica nanoshells with controllable size and porosity, stable and uniform walls, which are useful for drug delivery and imaging materials. In particular, the disclosure provides method of generating biodegradable iron doped silica nanoshells that can be size modified and porosity modified by changing, for example, the starting ratios of alkoxysilanes (e.g., RSi(OR′)₃-trialkoxysilanes, R₂Si(OR′)₂-dialkoxysilanes, R₃Si(OR′)-monoalkoxysilane) and optionally iron (III) ethoxide, by varying the speed of mixture or reaction time, or by varying the polystyrene template size or concentration.

Fe—SiO₂ nanoshells are synthesized by performing a sol-gel reaction with tetramethyl orthosilicate (TMOS) and iron ethoxide on an amino-polystyrene template. The particles are extremely mechanically stable which is beneficial for storage or post synthetic modification; however, this makes them non-ideal for ultrasound applications. Contrast signals from the particles are generated by shattering the nanoshells due to the rapid expansion of the perfluorocarbon gas/liquid within the particles under ultrasonic excitation which creates a de-correlation in sequential ultrasound pulses. Due to the high degree of mechanical stability of the nanoshells, only a small fraction of particles are actually imaged by ultrasound. Thus, the disclosure provides methods and compositions to increase the efficiency of the nanoshells as an ultrasound contrast agent. The method comprises mechanically weakening the hard silica shell of the particles. However, reducing the amount of TMOS used to synthesize the nanoshells does not usually result in weaker particles with thinner shells, but instead results in fragmented or fractured shells. This is most likely due to the model in which the nanoshells undergo an “island” like assembly process where by smaller colloids of polymerized siloxane assemble on the template surface rather than the TMOS polymerizing directly on the surface the template in a layer-by-layer fashion. Dopants such as trimethyl borate (TMB) which can polymerize into the siloxane network have been demonstrated to improve the mechanical stability of the shell. Thus, it was contemplated that introducing a destabilizing modification (e.g., an impurity) into the shell, which would copolymerize with tetramethyl orthosilicate, would render the shell mechanical weaker by creating holes or pockets in the silica network.

The disclosure provides a method and the resulting compositions whereby the mechanical strength of the silica shell is modified by introducing bulky R-groups associated with alkoxysilanes. For example, by using trialkoxysilanes that have bulky organic R-groups that polymerize with TMOS, will provide a shell, wherein the R-groups cannot withstand the calcination process at 550 C and thus leave “voids” or “pores” in the shell. This creates angstrom-nanometer holes/pockets/pores throughout the silica shell which will make it more brittle and likely to fracture under ultrasonic excitation. Particles have been successfully synthesized with various R-group substitutions by using a stoichiometric substitution based on the amount of silicon present in the original starting silane.

In one embodiment, a method of making a hollow silica nanoshell comprises (a) depositing a silica-shell precursor comprising a substituted alkoxysilane and, optionally, iron (III) ethoxide on a polyamino acid or polyamine functionalized nanotemplate particle to give core-shell spheres, wherein said polyamino acid or polyamine can comprise a homopolymer of an amino acid or an aliphatic amine with primary amine groups on the polymer backbone; (b) removing the template particle by calcination or using organic solvent to provide a hollow silica sphere having a porous silica nanoshell, wherein the size of pores in the nanoshell are defined by the substituted side group of the alkoxysilane (e.g., tri-, di- or monoalkoxysilane).

The disclosure also provides a nanostructure including an intermediate in the production of a hollow nanostructure (e.g., nanoshells) comprising a degradable nanotemplate comprising a polyamine or polycarboxylic acid functionalized surface layer; and a layer of alkyl substituted alkoxysilane and, optionally, iron (III) ethoxide. As mentioned above, the nanotemplate can be any degradable material upon which an amination, polyamine-cationic coating can be applied, followed by coating the template with an alkyl substituted alkoxysilane. For example, the nanotemplate can be a polystyrene bead. Such polystyrene beads are easily synthesized and their sizes can be easily modified. The nanotemplate typically has a nanometer cross section (e.g., diameter, width etc.). For example, the nanotemplate can be substantially sphere-shaped and have a diameter of about 10 nm to 3 μm in diameter. The template is coated with a silica material. In one embodiment, a layer of a compound comprising the structure of Formula I is coated on the template:

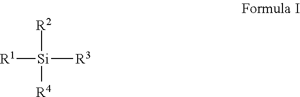

wherein, R¹-R⁴ are independently selected from the group consisting of H, D, optionally substituted (C₁-C₁₈)alkyl, optionally substituted (C₁-C₁₈)alkenyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether; wherein at least one of R¹-R⁴ is an optionally substituted alkoxy, and wherein if three of R¹-R⁴ are methoxy groups then the fourth R group is selected from the group consisting of D, optionally substituted (C₁-C₁₈)alkyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted (C₂-C₁₈)alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether. In another embodiment, at least two of R¹-R⁴ are optionally substituted alkoxy groups. In another embodiment, at least three of R¹-R⁴ are optionally substituted alkoxy groups. In yet a further embodiment, R¹-R³ are optionally substituted alkoxy groups, and R⁴ is an optionally substituted (C₂-C₁₈)alkoxy group. For example, an alkyl substituted alkoxysilane can be used having the general structure of formula II:

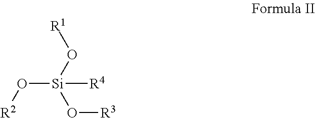

wherein R¹, R² and R³ are optionally substituted alkyls and wherein R⁴ is independently an optionally substituted alkyl. In another embodiment, R¹-R⁴ are independently selected from the group comprising optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted cycloalkenyl, optionally substituted aryl. The alkyl may be a halo substituted alkyl. For example, in one embodiment, R¹, R² and R³ are halo substituted alkyls. In another embodiment, the halo substitution can a fluorine (e.g., a fluorinated alkyl). In one embodiment, when the trialkoxysilane is a triethoxysilane or a trimethoxysylane, then R⁴ is a C₂-C₁₈ optionally substituted alkyl. As described above, the disclosure provides methods of modifying porosity by modifying the size or R4 of a trialkoxysilane as set forth in the formula above. Thus, an intermediate of the disclosure can be generated with a desired R4 group (based upon the size of a pore or the fragility of the nanoshell) and upon removal of the nanotemplate via calcination or other removal of the template a nanoshell having a desired porosity will be obtained.

The silane used to introduce the “impurity” into the siloxane network may be a tri-, di- or mono-alkoxysilane with multiple substituted R-groups sufficient to generate larger or differently structured pockets or pores. For example, using the general methods describe above and elsewhere herein, nanoshells were synthesized using a 1:1.7 molar ratio of pentafluorophenyl triethoxysilane to tetramethyl orthosilicate. Furthermore, continuous imaging lifetime of gas filled silica particles at maximum mechanical index had previously been approximately 15 minutes. The particles that have been substituted with the pentafluorophenyl triethoxysilane have an imaging lifetime at maximum MI well over two hours as the Doppler signal continued to persist.

In addition, Fe—SiO₂ nanoshells as described below are being developed as a High Intensity Focused Ultrasound (HIFU) sensitizing agent as well as mechanical ablative agent. Under HIFU excitation, perfluorocarbon (PFC) (liquid or gas) filled nanoshells undergo cavitation which is sufficiently destructive to mechanically damage and liquefy tissue; this destruction is contained within the focal volume of the HIFU transducer applying the ultrasonic force. It has been observed that different surface functionalization of the nanoshell surface are capable of affecting the HIFU threshold necessary for cavitation. Thus, modifying the “wettability” of the nanoshell surface by using, for example, highly fluorous functional groups, can modify the necessary energy for the perfluorocarbon gas/liquid within the particle to expand through and shatter the silica shell. As can be seen from Table 1, different degrees of functionalization with various fluorous silanes results in measurably different responses both in minimum mechanical index imaging threshold and the necessary output power for HIFU. The “% particle mass added” refers to the amount of fluoro-silane added relative to the particle mass to functionalize the particles. From the results in Table 1 it is suggested that different functionalizations on the particle surface results in different effects under ultrasound, both increasing or decreasing the thresholds for imaging and HIFU dependent on the structure and quantity of the R group functionalizing the particle surface.

TABLE 1

| | % Particle | MI | HIFU % |
|---|---|---|---|
| | Mass | Thresh- | Output |
| Fluoro-Silane Added | Added | old | Threshold |
| None | None | 0.64 | 32 |
| (heptafluoroisopropoxy)pro- | 1 | 0.52 | 18 |
| pyltriethoxysilane | | | |
| (heptafluoroisopropoxy)pro- | 5 | 0.42 | 30 |
| pyltriethoxysilane | | | |
| (heptafluoroisopropoxy)pro- | 10 | 0.42 | 45 |
| pyltriethoxysilane | | | |
| trimethoxy(trifluroisopropyl)silane | 1 | 0.56 | 36 |
| trimethoxy(trifluroisopropyl)silane | 5 | 0.7 | 30 |
| trimethoxy(trifluroisopropyl)silane | 10 | 0.7 | 25 |

In one embodiment, the disclosure provides a hollow silica sphere made from a silicon-containing compound with silicon atoms derived from, for example, mono-, di-, tri and tetra-alkoxysilanes, silicic acid, sodium silicate and the like. For example, any number of commercially available alkoxysilanes can be used (see, e.g., http:[//][www.]gelest.com/GELEST/Forms/GeneralPages/prod_list.aspx)?pltype=1&classtype=silanes&alpha=65; (brackets introduced to eliminate the hyperlink); the list of alkoxysilanes is quite extensive and can easily be identified in the art). For example, a brief but non-exhaustive list includes, (3-acetamidopropyl)trimethoxysilane, 2[acetoxy(polyethyleneoxy)propyl]triethoxysilane, acetoxyethyltriethoxysilane, acetoxyethyltriemethoxysilane, acetoxymethyltriethoxylsilane, acetoxymethyltrimethoxysilane, 3-acetoxypropylmethyldimethoxysilane, 3-acetoxypropyltrimethoxysilane, 3-(N-acetyl-4-hydroxyprolyloxy)propyltriethoxysilane, N-(acetylglycyl)-3-aminopropyltrimethoxysilane, N—(N-acetylleucyl)-3-aminopropyltriethoxysilane, 3-acrylamidopropyltrimethoxysilane, 3-acrylamidopropyltris(trimethylsiloxy)silane, N-(3-acryloxy-2-hydroxypropyl)-3-aminopropyltriethoxysilane, (acryloxymethyl)phenethyltrimethoxysilane, acryloxymethyltrimethoxysilane, (3-acryloxypropyl) dimethylmethoxysilane, (3-acryloxypropyl)methylbis (trimethylsiloxy)silane, (3-acryloxypropyl)methyldiethoxysilane, (3-acryloxypropyl)methyldimethoxysilane, (3-acryloxypropyl) trimethoxysilane, (3-acryloxypropyl)trimethoxysilane, N-allyl-aza-2,2-dimethoxysilacyclopentane, 3-(N-allylamino) propyltrimethoxysilane, allyldimethoxysilane, allylmethyldimethoxysilane, 11-allyloxyundecyltrimethoxysilane, m-allylphenylpropyltriethoxysilane, allyltriethoxysilane, allyltrimethoxysilane, 4-amino-3,3-dimethylbutylmethyldimethoxysilane, N-3-[amino(polypropylenoxy)] aminopropyltrimethoxysilane, 4-aminobutyltriethoxysilane, N-(2-aminoethyl)-11-aminoundecyltrimethoxysilane, N-(2-aminoethyl)-3-aminoisobutyldimethylmethoxysilane, N-(2-aminoethyl)-3-aminoisobutylmethyldimethoxysilane, N-(2-aminoethyl)-3-aminopropylmethyldiethoxysilane, N-(2-aminoethyl)-3-aminopropylmethyldimethoxysilane, N-(2-aminoethyl)-3-aminopropyltriethoxysilane, N-(2-aminoethyl)-3-aminopropyltrimethoxysilane, (aminoethylaminomethyl) phenethyltrimethoxysilane, N-(6-aminohexyl) aminomethyltriethoxysilane, N-(6-aminohexyl) aminopropyltrimethoxysilane, 3-(m-aminophenoxy) propyltrimethoxysilane, m-aminophenyltrimethoxysilane, p-aminophenyltrimethoxysilane, 3-aminopropyldiisopropyl ethoxysilane, 3-aminopropyldimethylethoxysilane, 3-aminopropylmethyldiethoxysilane, 3-aminopropyltriethoxysilane, 3-aminopropyltriethoxysilane, 3-aminopropyltrimethoxysilane, 3-aminopropyltris(methoxyethoxyethoxy)silane, 11-aminoundecyltriethoxysilane, (azidomethyl) phenethyltrimethoxysilane, p-azidomethylphenyltrimethoxysilane, 3-azidopropyltriethoxysilane, 4-(azidosulfonyl) phenethyltrimethoxysilane, 6-azidosulfonylhexyltriethoxysilane, and 11-azidoundecyltrimethoxysilane. The disclosure can include many other tetraalkoxysilanes, trialkoxysilanes, dialkoxysilanes or monoalkoxysilanes to introduce defects in the silica network. In one embodiment, the tetraalkoxysilanes is mixed with iron (III) ethoxide to generate a doped iron silica nanoshell. In one embodiment, the silicon-containing compound is hydrolyzed under acidic conditions before it reacts to form a silica shell.

The disclosure further provides a method for synthesis of hollow silica spheres. Commercial polystyrene or latex beads and their polyamine or polycarboxylate functionalized derivatives can be used in the disclosure as templates. The polymer core template used in the disclosure can have a narrow size distribution and can be chosen from about 10 nm to about 3 μm (typically about 20-40, 40-60, 80-100 nm, 200 nm, 300 nm, 400 nm, 500 nm, 1000 nm, but may be larger). A polyamino acid (e.g., poly-L-lysine), or any other polyamine, can be used in the disclosure with the core template mixture. A silicon-containing compound (as described in the foregoing paragraph) alone or mixed with iron (III) ethoxide is added to react under conditions that cause the deposition of a silica gel shell on the polystyrene beads to form a uniform silica layer on the template. The polystyrene core and the polyamine layer is then removed by calcination or solvent extraction. Both methods of core removal provide a hollow silica sphere with a uniform, porous, stable silica shell.

The polystyrene beads and the polystyrene or latex beads with polyamine or polycarboxylate functionalized surfaces (not monoamine functionalized), which are used in disclosure, can be purchased from Polysciences Inc. and Invitrogen Co. The size of templates can be 10 nm, 20 nm, 30 nm, 45 nm, 80 nm, 100 nm, 200 nm, 500 nm, 750 nm, 1000 nm, or 2000 nm and both smaller and larger sized templates can be used (e.g., from about 10 nm to 2000 nm) as many are available via emulsion polymerization. These beads are monodisperse microspheres and are commercially packaged as 2.0-4.0% solids (w/v) aqueous suspensions. These sizes typically vary by about 10% from batch to batch of manufacturer. After coating using the methods of the disclosure the size increases by 10-15 nm, but solvent washing shrinks them slightly and those that are calcined shrink more. The larger ones tend to shrink more than smaller ones. This occurs due to partial dehydration, as the shell initially forms as a silica gel coating and on removal of water dehydration to silica of varying degrees of hydration occurs. After calcining they comprise rigid hollow balls of porous silica gel that undergo no further or limited size change.

The disclosure provides for the use of polyamine or polyamino acid coated templates, which gives a high yield of well-formed spheres. The polyamines used in disclosure are homopolymers of amino acids or aliphatic amines with primary amine groups on the polymer backbone. Such polyamino acids are poly-L-lysine, poly-L-arginine, and polyornithine, including solids or their aqueous solution. One type of homopolymer of aliphatic amine is polythyleneimine. The polystyrene beads or latex beads themselves having monoamines can template the deposition of a silica shell. The concentration of polyamino acids used in the disclosure is kept at low levels to avoid the formation of solid silica spheres templated by polyamino acids alone, which occurs at higher polyamino acid concentrations.

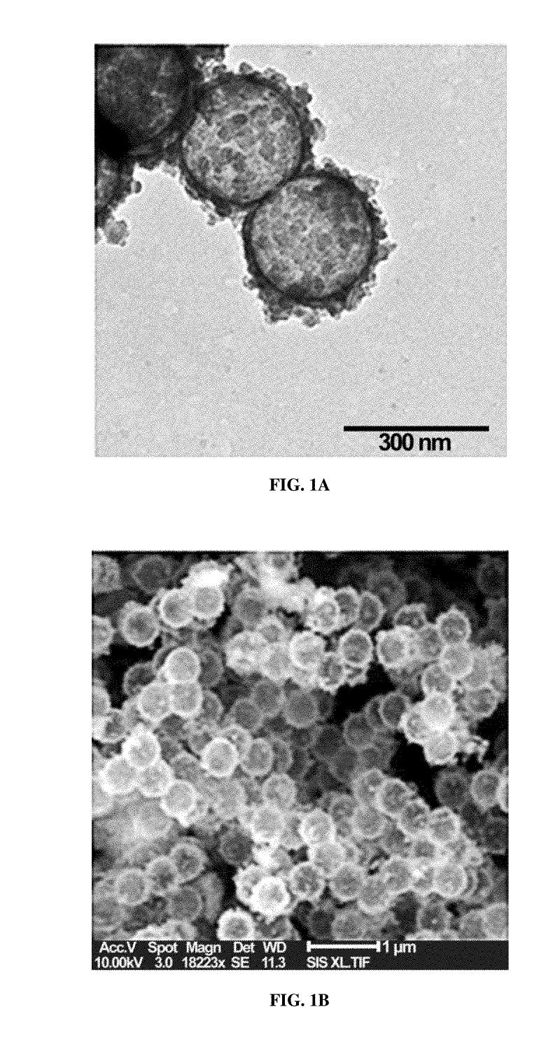

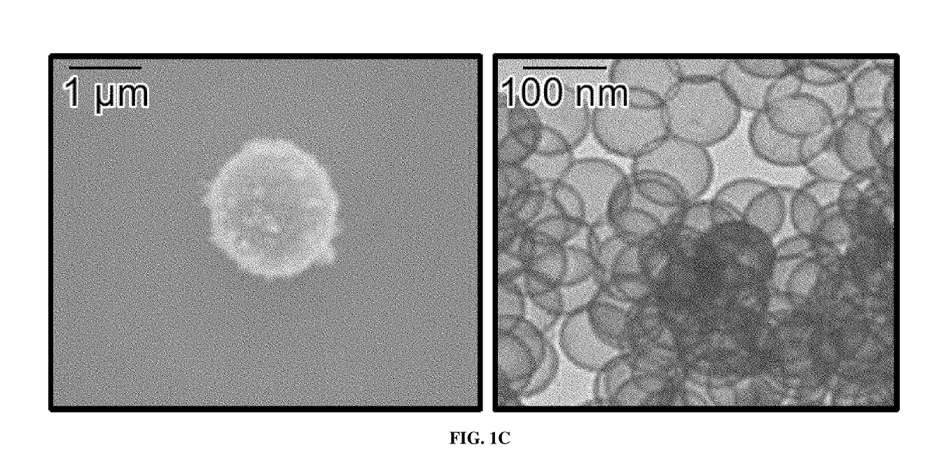

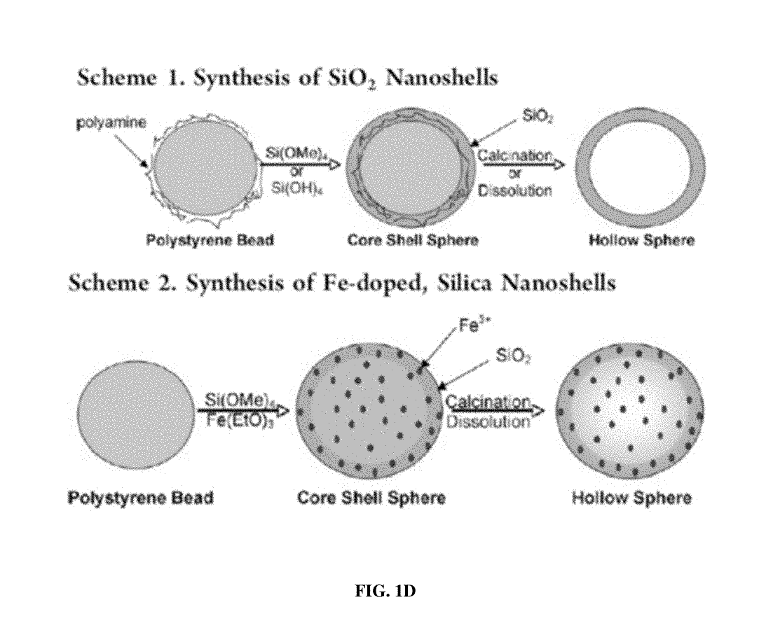

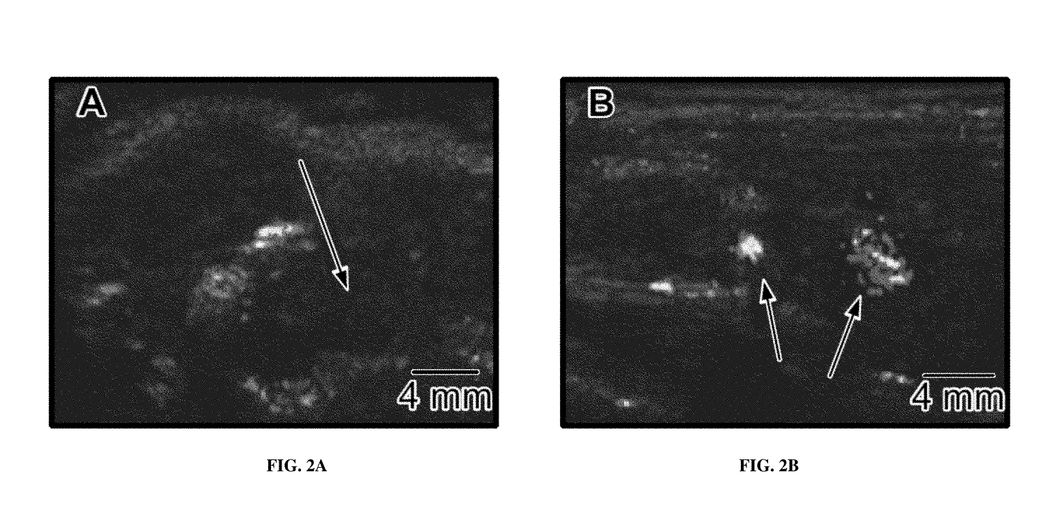

As shown in FIG. 1D, the polyamine functionalized polystyrene beads form shells of silica and iron.

As in the sketch of scheme 1 of FIG. 1D, the polystyrene or latex beads are mixed with polyamino acids or polyamine coated templates before the hydrolyzed silica-precursor (e.g., tetraalkoxysilane) solution is added. The dispersion of beads and 0.1% w/v polyamino acid aqueous solution are added to a phosphate buffer. The ratio of 0.1% w/v polyamino acids and the 2.75% w/v polystyrene beads is from 1:1 to 10:1 v/v and typically about 4:1. The final concentration of the polystyrene beads in the buffer solution is from 1:1000 to 1:10000 w/v but typically about 1:670 w/v.

One method of the disclosure is depicted in scheme 2 of FIG. 1D. As shown in scheme 2, the silica-shell precursor (e.g., tetraalkoxysilane) and iron (III) ethoxide is added to a mixture of amine coated or functionalized polystyrene or latex beads. By selecting appropriate reaction conditions such as temperature, pH, ratios and reaction time the polycondensation occurs and a silica-iron oxide gel shell is deposited on the polystyrene beads. The core-shell spheres are collected, washed and calcined at high temperature to remove the polymer core to give hollow silica-iron spheres. It will be recognized that the addition of iron (III) ethoxide is optional and is provided to improve biodegradability of the shells over time. Additionally, the inclusion of iron (III) ethoxide can be used to modify the ultrasound properties and imagine lifetime of silica nanoshells.

The template particles can be, for example, a latex or polystyrene bead. The template particle is then treated to comprise a polyamino acid or polyamine group. The template particles may also be purchased pre-functionalized with amine surface groups. The polyamino acid or polyamine group facilitate silica deposition. A silica shell is then deposited on the template. In one aspect, the template nanostructure is degraded to provide a hollow nanostructure of the disclosure. In other embodiments, the template nanostructure remains intact.

The nanostructures may be used with or without decomposing the template material. Batch fabrication is straightforward. The characteristics of the resulting hollow sphere make the nanostructures useful for application in molecular medicine and in ultrasensitive Raman, biomolecular, cellular imaging, and ultrasonic imaging.

Various polymers may be used as the template nanostructure in the generation of a nanostructure of the disclosure. For example, o-polyacrylamide and poly(vinyl chloride), poly(vinyl chloride) carboxylated, polystyrene, polypropylene and poly(vinyl chloride-co-vinyl acetate co-vinyl) alcohols, may be used.

The ready availability of monosized polystyrene spheres between 40 and 3000 nm provide a mass produced template for the high yield synthesis of mono-dispersed hollow silica-NPs with porous shell walls. The polymer spheres readily adsorb a monolayer of poly-L-lysine and other amino polymers in aqueous solution, which then serve as a basic catalyst coating for the gelation of silicic acid and alkoxysilanes.

The reaction is typically conducted at room temperature. The final concentration of hydrolyzed tri- or alkoxysilane in the reaction system is from about 10⁻³ M to 5×10⁻³ M and typically about 2×10⁻³ M. A useful concentration of hydrolyzed alkoxysilane provides a uniform and stable silica shell around the templates with narrow size distribution range, and in high yield based on the template. Higher concentrations of hydrolyzed alkoxysilane do not give a significantly thicker silica shells, but yield solid silica colloids as byproducts, which can have an irregular shape dependent on reaction conditions. The alkoxysilane does not need to be hydrolyzed for shell formation, but hydrolyzing the alkoxysilanes does increase the rate of shell formation.

The core-shell spheres can be isolated from solution by centrifugation. The precipitate can be washed by being dispersed in deionized water and centrifuged. These procedures are followed by washing the spheres with ethanol. These washing procedures in the disclosure are to remove excess reactant and phosphate buffer and are optional. After collection of the pure core-shell spheres by centrifugation, the polystyrene core can be removed, although it may not be desirable depending upon further processing or intended use.

Various methods can be used to remove the core nanotemplate structure. Two such methods that can be used to remove the polystyrene core are calcination and dissolution, preferably the method of calcination. To remove the core by dissolution, the core-shell precipitate is suspended in toluene or other solvent and the mixture is stirred 1 hour at room temperature and then collected by centrifugation. The washing procedure is repeated three more times and then the hollow spheres are washed twice with ethanol. The first solvent used in this step may be extended to dichloromethane, chloroform, ethylene diamine, tetrahydrofuran, dimethylformamide, or other solvents for the polymer core. The final product of the disclosure is obtained by drying the final pellet (e.g., at 60° C. under vacuum for 48 hours). To remove the polystyrene cores by calcinations, the core-shell spheres are dried at room temperature overnight until the core-shell particles form a fine powder, and then heated in air at 400-900° C. for 3-18 hours, typically heating at 550° C. for 18 hours. Temperature ramp and decline rates are from 0.1° C./min to 10° C./min, and are typically 1° C./min.

The nanoshells described above can be used in various ultrasound methods for imaging and treatment. For example, the ultrasound imaging of silica nanoshells are rigid and would not be expected to respond non-linearly under ultrasound; however, the shells have been designed to fracture at a given US pressure to not only create a signal with Doppler imaging, but then release perfluorocarbon gas that is able to expand and contract will generate non-linear signals until it dissolves. This design allows for the particles to be imaged not only by Doppler modalities but also by contrast specific imaging modalities such as contrast pulse sequencing (CPS) imaging and harmonic imaging. For both imaging types, it is possible to generate signal because of the substantial acoustic impedance mismatch between the gas and the surrounding environment.

PFC gas filled degradable nanoshells can attenuate ultrasound energy to increase the local heat deposited in the region of the nanoshells which can thermally ablate tissue. A secondary modality of ablation is also generated by the nanoshell interaction with ultrasound in the form of nanoshell cavitation causing mechanical damage of the tissue leading to mechanical ablation. By optimizing the ultrasound power and the shell thickness, the cavitation component can be the dominant response thereby making the tissue destruction highly localized.

Furthermore, experiments described below show that nanoshells are able to be filled with perfluorocarbon liquids. This liquid filling enables a new application in which high power ultrasound converts the nanoshells to 1 mm gas bubbles via coalescence. This enables the particles to be used to occlude the vascular supply of a tumor. Data has been obtained in vitro showing that the liquid filled nanoshells can be converted to 1 mm gas bubbles using high power ultrasound.

In one embodiment, nanoshell enhanced HIFU in combination with viral therapy/liposomal or polymeric formulations/chemotherapeutic agents (Local Administration) can be performed. For example, after a local injection of perfluorocarbon liquid or gas filled nanoshells of the disclosure mechanical cavitation is used to liquefy the tissue at the injection site. The liquefied tissue can then be removed with vacuum to leave behind a cavity which can then be refilled with a variety of formulations. The cavity filled with any number of different therapeutics acts a drug reservoir for long term drug release and retention specifically in the site of diseased tissue such as cancers, fibroids and other abnormal growths where surgery may not provide optimal therapy. The therapeutic added to the cavity can also be an oncolytic virus, a liposomal encapsulated virus, a liposomal formulation, or a polymeric formulation of a chemotherapy agent which would benefit from slow release in the vicinity of the tumor foci; the use of 100% cavitational HIFU or high percent cavitational HIFU would allow precise control over the volume of tissue to be ablated. The inflammatory response which typically follows ablative therapies may enhance either oncolytic virus, antibody therapy, or chemotherapeutic drug delivery and retention specifically in the site of focal ablation. This may be ideal for treating cancers, fibroids and other abnormal growths where surgery may not provide optimal therapy.

In another embodiment, a nanoshell and HIFU “Tissue drilling” process is used. After a local injection of perfluorocarbon liquid or gas filled nanoshells of the disclosure, mechanical cavitation is used to liquefy the tissue at the injection site. Removing this liquefied tissue with suction can create a cavity which can then be refilled with additional nanoshells that can be used to further enlarge or deepen a cavity to rapidly ablate larger tissue volumes.

In another embodiment, long term imaging markers are provided. In this embodiment, perflurocarbon liquid filled nanoshells, functionalized with a fluorinated alkoxysilane are used for long term in vivo ultrasound imaging. The fluorinated alkoxysilane makes the particles “non-wetting.” This will prevent PFC from escaping the particles and also particle degradation.

A nanoshell (optionally liquid or gas filled) of the disclosure can be formulated with a pharmaceutically acceptable carrier suitable for delivery to a subject, although the nanostructure may be administered alone, as a pharmaceutical composition.

A pharmaceutical composition according to the disclosure can be prepared to include a nanostructure of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “effective dose” is meant the quantity of a nanostructure according to the disclosure to sufficiently provide measurable SERS signals. Amounts effective for this use will, of course, depend on the tissue and tissue depth, route of delivery and the like.

Typically, dosages used in vitro may provide useful guidance in the amounts useful for administration of the pharmaceutical composition, and animal models may be used to determine effective dosages for specific in vivo techniques. Various considerations are described, e.g., in Langer, Science, 249: 1527, (1990); Gilman et al. (eds.) (1990), each of which is herein incorporated by reference.

As used herein, “administering an effective amount” is intended to include methods of giving or applying a pharmaceutical composition of the disclosure to a subject that allow the composition to perform its intended function.

The pharmaceutical composition can be administered in a convenient manner, such as by injection (e.g., subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit.

For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Supplementary active compounds can also be incorporated into the compositions.

The following examples are intended to illustrate but not limit the disclosure. While they are typical of those that might be used, other procedures known to those skilled in the art may alternatively be used.

### EXAMPLES

### Example 1

Preparation of the solution of hydrolyzed tetramethoxysilane. 14.0 mL tetramethoxysilane is added to 100 mL 0.01 M hydrochloric acid. The mixture is stirred at room temperature for 15 minutes. The solution is to be used as the precursor to deposit silica shells directly.

### Example 2

Synthesis of core-shell silica spheres with 100 nm amine polystyrene beads. 4.0 mL of 2.6% w/v 100 nm sized amine polystyrene beads, 16 mL of 0.1% poly-L-lysine solution and 75 mL of 0.1 M phosphate buffer are mixed in a 150 mL pear-shaped flask. 2 mL of hydrolyzed tetramethoxysilane is added and the mixture is stirred vigorously with a vortex agitator at a speed of 3000 rpm. The stirring lasts 5 minutes at room temperature and the mixture is transferred into two 50 mL centrifuge tubes. A white precipitate is collected by centrifugation. The core-shell particles are suspended in deionzed water and stirred with the vortex agitator for 5 minutes and then spun down again by centrifugation. The washing procedure is repeated one more time followed by washing with ethanol. 185 mg Core-shell particles are dried in vacuum at 60° C. for 48 hours.

### Example 3

Synthesis of core-shell silica spheres with 200 nm amine polystyrene beads. The synthesis procedure is similar to example 2, except that the 100 nm amine polystyrene beads are replaced by 200 nm amine polystyrene beads.

### Example 4

Synthesis of titania core-shell silica spheres with 200 nm amine polystyrene beads. 2.0 mL of 2.6% w/v 100 nm sized amine functionalized polystyrene beads and 80 mL of absolute ethanol are mixed in a 150 mL pear-shaped flask. 2.0 mL of 1 M titanium t-butoxide/ethanol solution is added and the mixture is stirred vigorously with a vortex agitator at a speed of 3000 rpm. The stirring lasts 2 minutes at room temperature and the mixture is transferred into two 50 mL centrifuge tubes. A white precipitate is collected by centrifugation. The core-shell particles are suspended in absolute ethanol and stirred with the vortex agitator for 5 minutes and then spun down again by centrifugation. The washing procedure is repeated one more. 120 mg Core-shell polystyrene/titania particles are dried in vacuum at 60° C. for 48 hours.

### Example 5

Removing polymer cores by calcination. 10 mg of dry core-shell silica particles are placed in a furnace and the temperature is raised at a speed of 5° C./min to 450° C. The core-shell particles are calcinated in air at 450° C. for 4 hours and the temperature is then cooled at a speed of 5° C./min until it reaches room temperature. 3.2 mg of final product of hollow silica spheres is obtained as a white powder. Yield is nearly quantitative based on the number of template spheres. Greater masses of dry core-shell silica particles are regularly calcined

### Example 6

Removing polymer cores by dissolution in toluene. 10 mg of dried core-shell spheres are suspended in 20 mL of toluene and the mixture is stirred with a magnetic stirrer for 1 hour. The solid is collected by centrifugation. The washing procedure is repeated three more times followed by drying the particles in vacuum at 60° C. for 48 hours. 3.7 mg of hollow silica spheres is obtained as white powder. Some residual polystyrene is still evident from the infra-red spectra. The TEM photographs of this material show thicker shell walls, presumably from adsorbed polystyrene on the silica walls.

### Example 7

Removing cores by dissolution with added ethylene diamine 10 mg of dried core-shell spheres are suspended in a mixture of 5 mL of ethylene diamine and 15 mL dichloromethane and stirred with a magnetic stirrer for 1 hour. The solid is collected by centrifugation. The washing procedure is repeated three more times followed by drying the particles in vacuum at 60° C. for 48 hours. 3.5 mg of hollow silica spheres is obtained as white powder. Compared to Example 5 nearly all the polystyrene core is removed by this method.

### Example 8

Functionalized the hollow silica spheres with 3-aminopropyl (trimethoxy)silane. 1 mg of calcined hollow silica spheres, prepared from the 100 nm templates, are suspended in 2 mL of 1% 3-aminopropyl(trimethoxy)silane acetone solution, The mixture is stirred slowly for 2 hours with a magnetic stirrer followed by collecting the particles by centrifugation. The collected particles are washed with ethanol and dried in vacuum for 24 hours at room temperature.

TABLE 2
Size of hollow silica spheres isolated and its
dependence on the size of the templates and the
methods of removing the polystyrene cores.

| Size of template (nm) | 100 | 200 | 500 |
|---|---|---|---|
| Diameter of core-shell spheres (nm) | 126± | 210 ± 6 | 454 ± 16 |
| Diameter of hollow spheres after | 126 ± 7 | 205 ± 7 | 443 ± 21 |
| calcinations (nm) | | | |
| Diameter of hollow spheres after | 102 ± 8 | 188 ± 9 | 397 ± 15 |
| dissolution (nm) | | | |

### Example 9

To prepare the iron (III) ethoxide solution 20 mg of iron (III) ethoxide is weighed out and suspended in 1 ml of anhydrous ethanol and then sonicated in a bath sonicator until a uniform brown translucent solution is observed (˜1-3 hours). The iron (III) ethoxide solution is then reserved in a desiccator until needed.

To begin the synthesis of iron-doped silica nanoshells, iron (III) ethoxide solution is placed in a bath sonicator for 90 minutes to dissolve any precipitate or nanocrystalline materials which may have formed. In an Eppendorf tube, 50 ul of amino-polystyrene templates are suspended in 1 ml of absolute ethanol. Then 10 ul of iron ethoxide solution and a size dependent amount of Tetramethyl orthosilicate is added (3.1 ul for 200 nm and smaller particles, 2.7 ul for larger than 200 nm particles). The solution is then mixed on a pulsing vortex at 3000 rpm for 5 or 6 hours depending on the size of the particles, 6 hours is necessary for the larger nanoshells to form completely. The particles are then pelleted on a centrifuge (speeds/times vary based on centrifuge used) and the supernatant is discarded. The particles are then resuspended in ethanol and then pelleted again, the supernatant is discarded. This step is repeated twice more to remove all excess and unreacted materials. The particles are then dried overnight at room temperature and calcined for 18 hours at 550 C. The particles are then stored dry in an Eppendorf tube.

Silica nanoshells can be synthesized in a highly reproducible manner by performing a sol-gel reaction on polystyrene templates using Silicic Acid, tetramethyl orthosilicate, tetraethyl orthosilicate, as well as various dopants as described above. Particles are then calcined which removes the polystyrene core leaving a dehydrated, rigid, and nanoporous shell. FIGS. 1A-1B contain transmission electron microscopy and scanning electron microscopy images of 500 nm iron doped silica nanoshells. FIG. 1C shows non-doped silica nanoshells. Note synthesis can readily include functionalization with coatings which have been shown to increase sticking (PEI) to all cells or endocytosis (folate) to cancer cells.

Perfluorocarbon Containing Nanoshells:

To fill the nanoshells with liquid, dried shells are evacuated in a Schlenk flask, the flask is filled with saturated perfluoropentane liquid, water is injected into the flask, and the solution is shaken to disperse the shells with entrapped liquid perfluoropentane. The perfluorocarbon liquid is contained for long periods (at least months) within the porous shell due to its extremely low solubility in water. In addition, the high surface tension of water may serve to seal the fluorous phase within the pores of the shell wall as water enters the outer surface of the porous shell by capillary action. The PFC liquid filled degradable nanoshells can be injected pre-operatively and can be retained at the site of injection to act as a local marker.

Local Intra-Operative Ultrasound Marker:

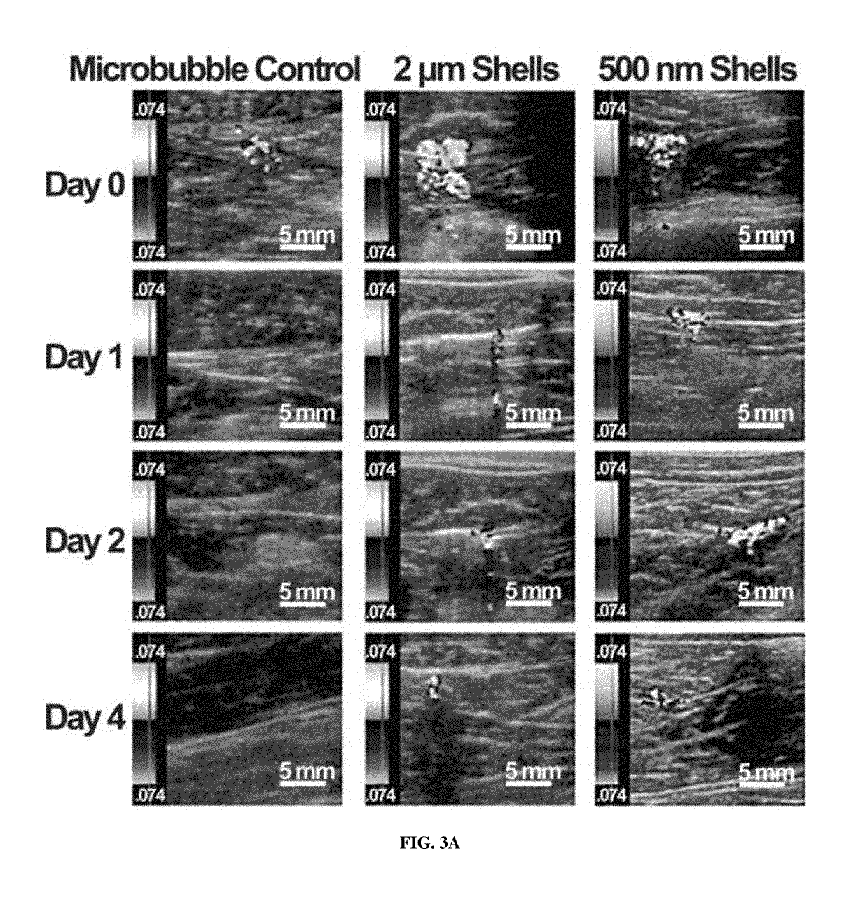

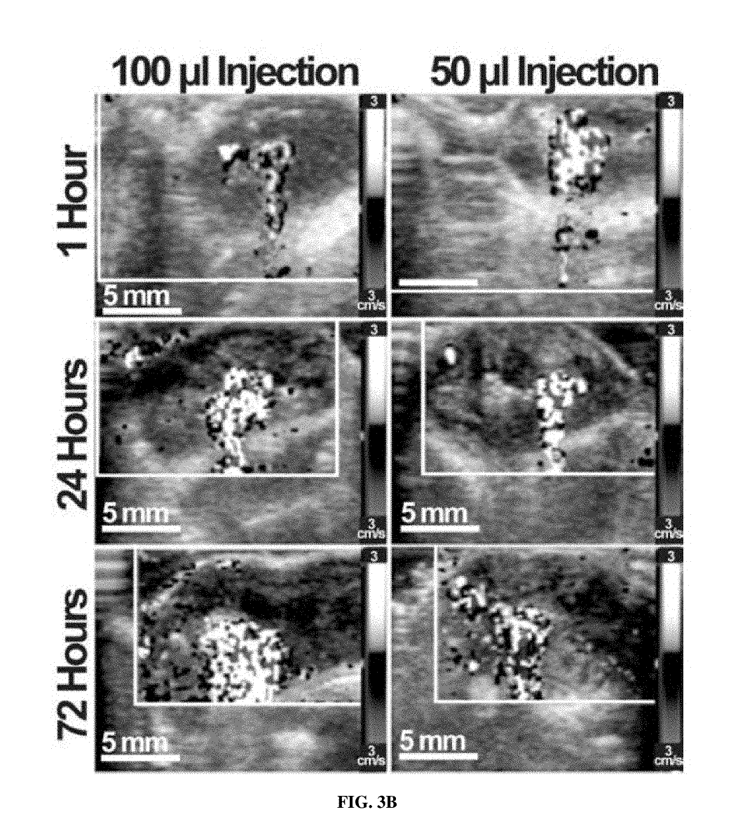

Extensive in vivo testing has been performed in this application. To fill the nanoshells with gas, dried shells are evacuated in a Schlenk flask, the flask is filled with saturated perfluoropentane vapor, water is injected into the flask, and the solution is shaken to disperse the shells. The perfluorocarbon vapor is contained within the porous shell due to its extremely low solubility in water. In addition, the high surface tension of water may serve to seal the fluorous phase within the pores of the shell wall as water enters the outer surface of the porous shell by capillary action. Gas-filled shells can be prepared in a dispersed state using ultrasonic agitation, and remain dispersed and retain gas for at least several weeks due to their surface charge. The gas filled degradable nanoshells can be injected pre-operatively and can be retained at the site of injection to act as a local marker. To examine the optimal dose of the nanoshells, a nude mouse model with PyVmT tumors grown in the mammary tissue with two tumors per mouse was employed. Mice were injected with 500 nm Fe—SiO₂-FITC nanoshells and imaged with color Doppler ultrasound after the initial injection and 1 hour, 24 hours, or 72 hours post injection. There is little qualitative difference in signal between 50 μl and 100 μl injections as shown in FIG. 3B. Moreover, there is very little difference seen after 72 hours indicating that the particles retain the perfluoropentane gas in the absence of imaging; this will allow patients to have the particles injected at least the day prior to surgery. In FIG. 3B, each of the images displayed are from different mice demonstrating the high degree of performance consistency and reproducibility of the 500 nm Fe-doped SiO₂ nanoshells.

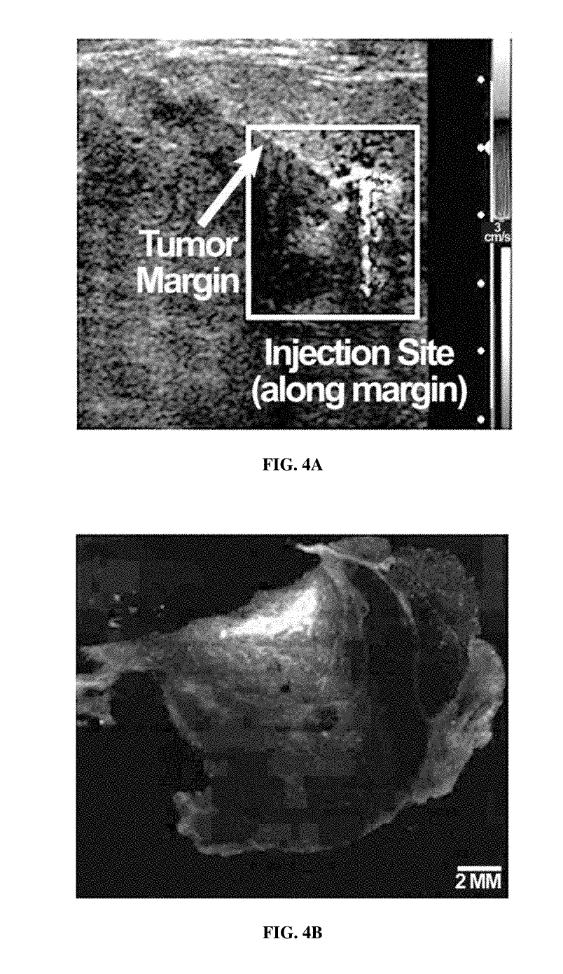

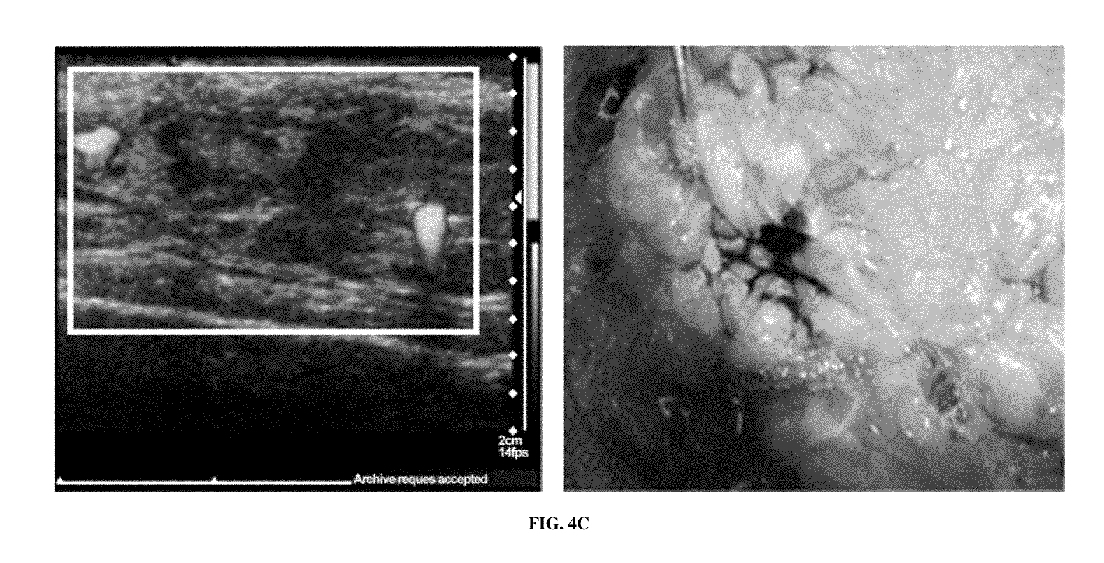

Additionally, nanoshells have been tested ex vivo in excised human mastectomy tissue. For application in breast conservation surgery, the goal is to pre-operatively inject these particles via CT guidance in the same fashion that radioactive seeds or guide wires are currently implanted to help precisely localize the tumor for excision. Therefore, it is desirable that the particles remain stationary prior to and throughout surgical excision. As shown in FIG. 4A, the particles can be precisely injected next to a tumor margin and will not be transported away from site of localization thereby enabling multiple injections around the tumor to more thoroughly outline the margin. FIG. 4B contains a fluorescent microscopy image of a cross-sectional cut from the injection site. The fluorescence is from FITC that was covalently linked to the surface of the particle; the fluorescence being restricted to an area of several square millimeters is consistent with the volume that was injected initially, which reconfirms that the particles are localized at the injection site.

Tumor Detection Via Systemic Injection:

Nanoshells are filled with gas as previously described or could be filled with liquid perfluorocarbon. In vivo CPS imaging was tested using a second type of systemic injection on two Nu/Nu mice with intraperitoneal IGROV-1 ovarian tumors. 200 μg of PFP filled 2 μm or 500 nm shells were diluted into 3 ml of saline and injected into the peritoneum (IP). The particles were imaged at high MI using CPS imaging intermittently over two hours. Intraperitoneal injections have been previously used for systemic delivery in murine models.

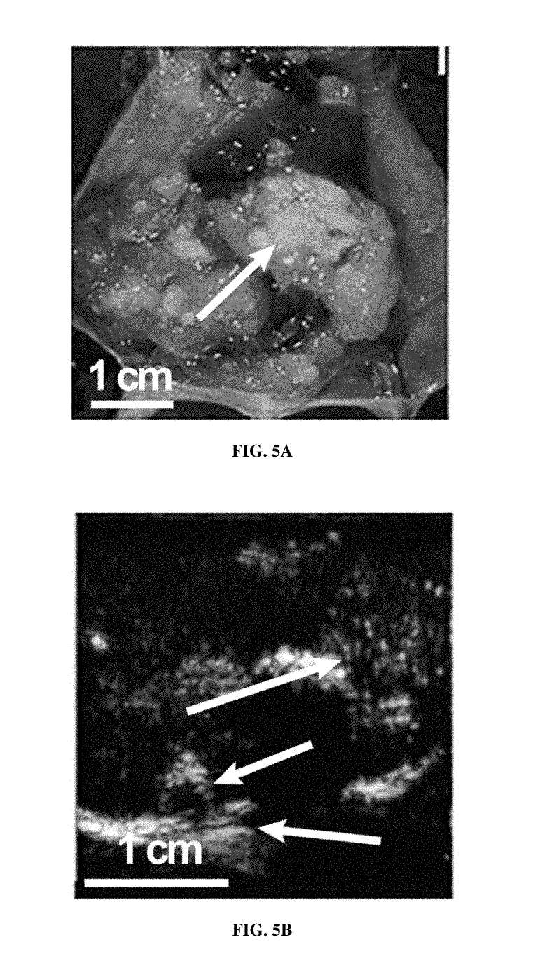

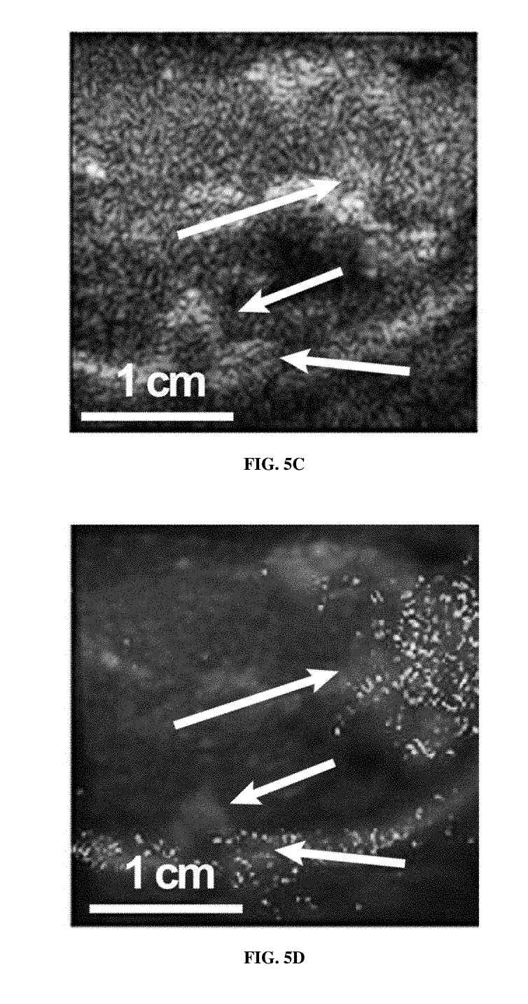

These mice had a late stage ˜1 cm tumor mass (FIG. 5A, red arrow). In FIG. 5B-D, the bottom boundary (blue arrow) is actually the bottom of the mouse and the mound like region on the bottom (green arrow) is the spinal column Image processing techniques were used to generate FIG. 5D which (a) corrects motion due to sonography and the breathing of the mouse, (b) selects the signal from single particles by taking the differences in intensity between a few consecutive frames, (c) integrates the particle signal from the entire sonography exam, and (d) displays the signal from the particles as a red-yellow heat map superimposed on the grey scale image. As shown in FIG. 5D, the signal generated by the particles could be seen specifically in the tumor 1 hour after injection. This illustrates the ability to readily visualize even the small signal from single event in vivo several hours post injection. Note this imaging is not possible with soft commercial CEUS bubbles because they do not stick to tumors and their lifetime in tissue and in circulation is so brief, they are unable to accumulate in tumors. Instead for imaging with soft commercial microbubbles, a bolus inject is typically employ and the perfusion kinetics into the tumor must be carefully measured.

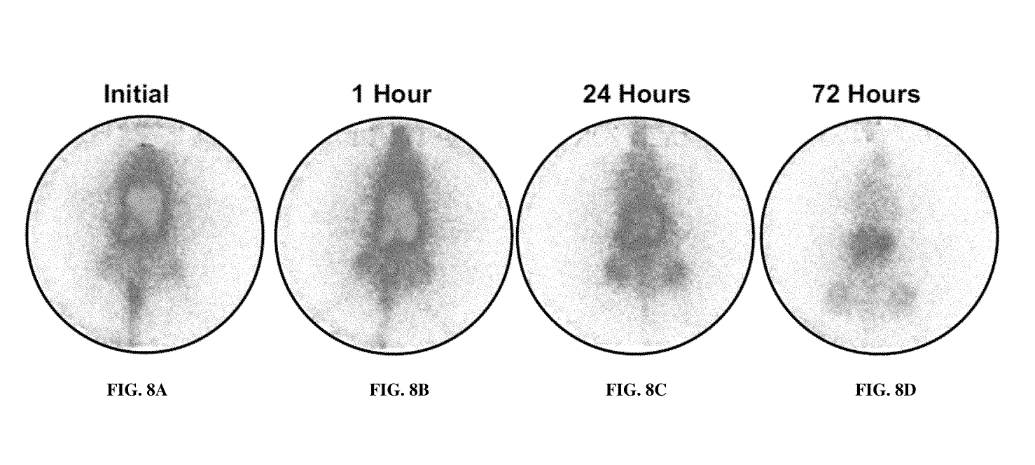

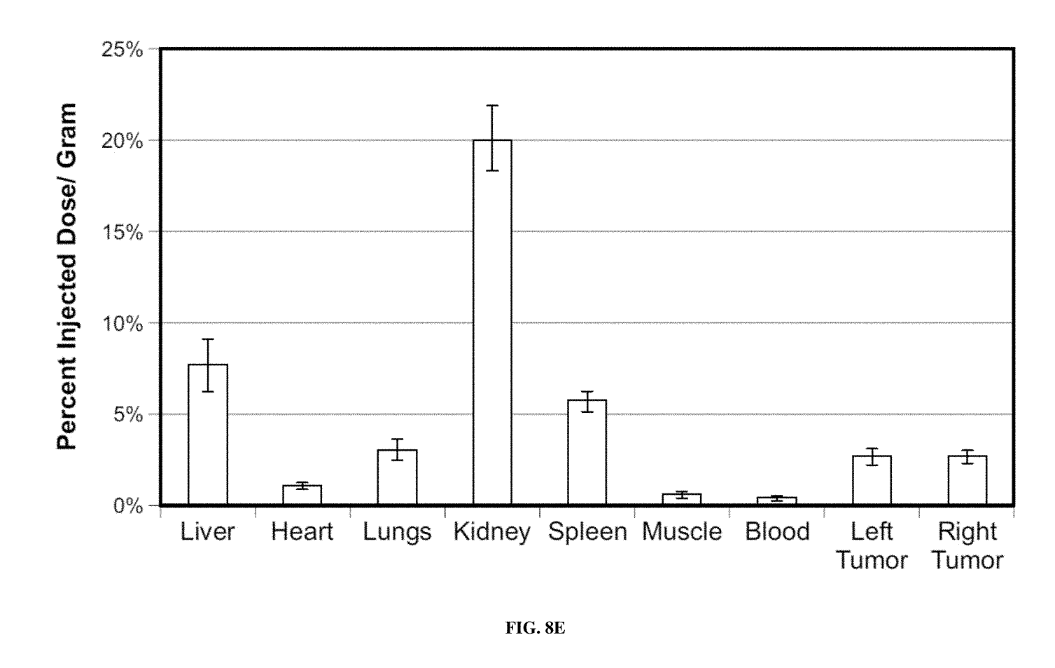

A biodistribution study was performed on healthy Nu/Nu mice with 500 nm gas filled degradable silica shells linked with DTPA (indium chelator) to show that particles remain in circulation for an hour and only accumulate in the liver; this shows there should be little background sticking of particles to other tissues thereby enable tumor localization. 2 mg of particles were radiolabeled with ˜100 μCi of In-111. Four nude mice were utilized for this experiment. Each mouse received a 100 μl IV injection via the tail vein. The mice were imaged by gamma scintigraphy during the initial injection and at 1, 24 and 72 hours post injection. After 72 hours, the mice were sacrificed, and the organs were harvested and deposited in a Gamma Counter where radioactivity level was measured. As can be seen from FIG. 8D, even after 72 hours, some signal is still detectable in the blood indicating that some particles are still in circulation, potentially allowing for long term imaging of tumor vasculature.

Nanoshell Enhanced Ultrasonic Ablation-Type 1:

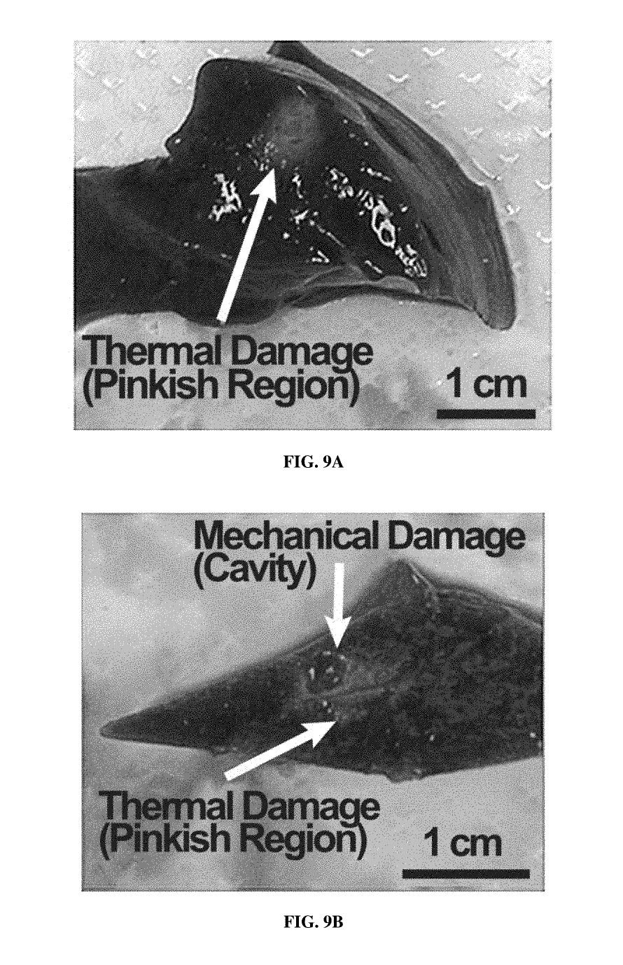

A set of animal experiments were performed to demonstrate that the gas filled particles could denature tissue via HIFU much faster the normal HIFU. Normal HIFU denatures tumors tissue via heating so long insonation times are required. The silica shell CEUS HIFU rapidly induces liquification of tissue via cavitation so the process is very fast while being highly localized. Four healthy New Zealand white rabbits (˜4 kg) were used to establish the feasibility of this mode of ablation. It was found that at a given power of ultrasound energy applied, using a continuous 800 KHz pure tone waveform with a peak negative pressure at 3 MPa, nanoshell enhancement could reduce the amount of time necessary to achieve a measurable response in tissue. As can be seen in FIG. 9A, highly energetic ultrasound alone can cause thermal damage in the liver after 60 seconds of exposure. However, an equally sized legion can be produced in 30 seconds with nanoshell enhancement with the addition of mechanical damage. Note outside the region of liquefaction there is a zone of thermal ablation showing that the nanoshells enhance both processes probably because the ultrasound is strong scattered by the liquefied tissue. This may be advantageous in tumor therapy since it would denature any cells near the liquification region thereby insuring no cancer cells escape from the tumor.

Nanoshell Enhanced Ultrasonic Ablation-Type 2:

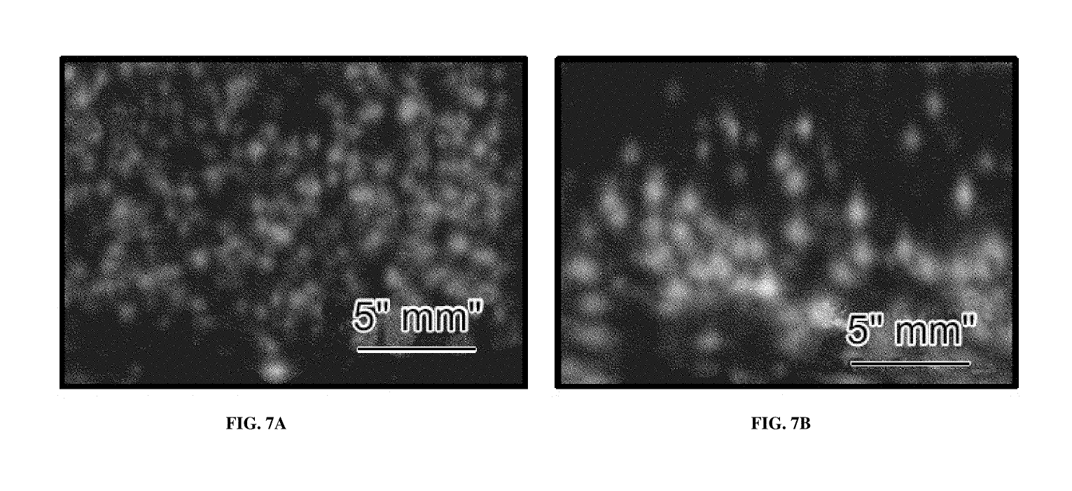

Perfluorocarbon liquid filling of nanoshells is accomplished by first evacuating the particles under vacuum in a vial and then with a syringe injecting liquid PFC into the vial. Then the solution is sonicated and water is added to the solution and further sonicated. The two solutions are immiscible, but no liquid separation phase is observed indicating that the liquid PFC is within the nanoshells. The conversion of liquid PFC within the nanoshells to gas and subsequent coalescence has been performed in vitro using a commercial diagnostic ultrasound machine. The nanoshells where suspended in an acoustically transparent container and then imaged at different mechanical indices (MI) using CPS imaging. As can be seen from FIG. 7A-B as the mechanical power of the ultrasound is raised from an MI of 0.97 to 1.9, a stimulated coalescence of approximately 1 mm bubbles is generated.

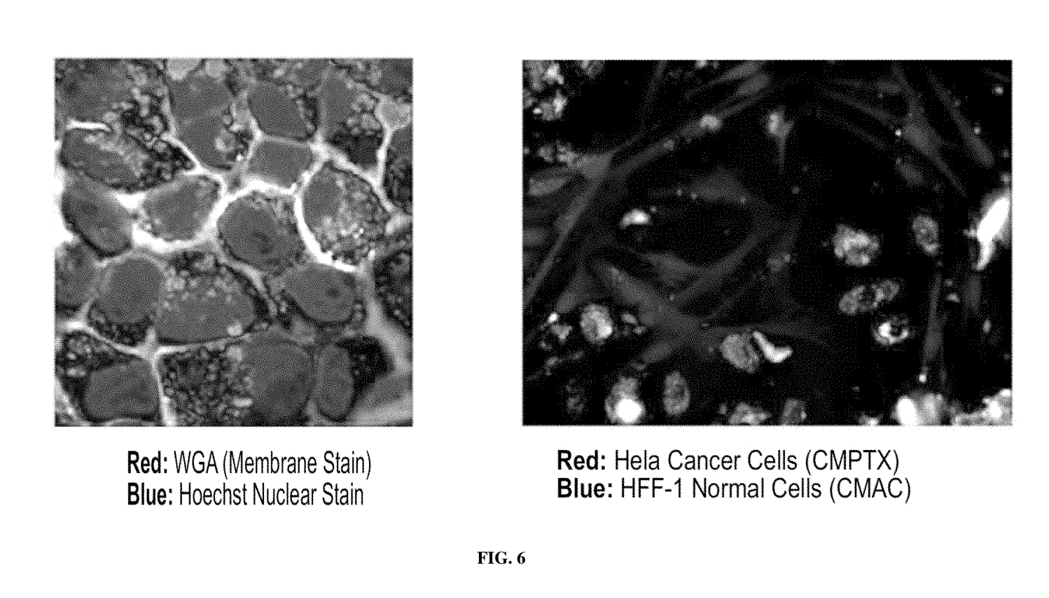

The surfaces of 100 nm silica NS have been functionalized with folic acid in order to specifically target and penetrate cancer cells. 3 mg of 100 nm hollow silica NPs were suspended in 1 mL absolute ethanol, followed by the addition of 0.3 uL of 3-aminopropylsilane for 1 hr in order to modify the NS surface with amines. Once the amine surface coating reaction was complete, the NPs were pelleted, washed twice in ethanol and once in DMSO, and the amine modified NS were then re-suspended in 1 mL of DMSO. To this suspension, 20 ug of fluorescein isothiocyanate (FITC) succinimidyl ester and different amounts of folic acid succinimidyl ester (2, 20, or 200 ug) were added and mixed together for 3 hours at room temperature. The succinimidyl ester disassociates allowing for the FITC and folate to bind to the amine coating. The FITC-Folate modified particles were collected by centrifugation and washed with DMSO and D. I. water before being re-suspended in 1 mL PBS for Dynamic Light Scattering (DLS) characterization and endocytosis experiments. With the use of fluorescent and confocal microscopy, it was found that as the amount of folate on the surface of the NS was increased, a higher amount of NS endocytose into HeLa cancer cells, a cervical cancer cell line (FIG. 6 left). The results show that with folate targeted NS particles as large as 327 nm in diameter can be obtained with relative ease and endocytosed by HeLa cells.

A cancer cell selectivity targeting experiment was performed using the silica NS functionalized with 20 ug FITC and 200 ug folate. HeLa cells and a normal cell line, Human Foreskin Fibroblast (HFF-1) were grown in separate flasks and then each of their cytoplasms were stained with a different color using one of Invitrogen CellTracker dyes. The two cell lines were then mixed together and incubated for 24 hrs in folate free media complete at 37° C. in a humidified atmosphere of 5% CO₂. Afterward, folate targeted NS were incubated with cells for additional 24 hrs. Subsequently, cells were washed 3× with DPBS to remove any excess NS, fixed with 4% PFA in DPBS solution, washed twice more with DPBS, and covered with Prolong Gold antifade reagent in order to prepare samples for visualization by fluorescent microscopy. Under fluorescent microscopy, it was found that the majority of the NS did interact more and tended to target the folate receptor rich HeLa cancer cells at a higher rate when compared to the HFF-1 normal cell line (FIG. 6 right).

Determine the Optimal Dose and Lifetime of Silica Nanoshells in a Mouse Model with IV Injection.

IGROV-1 ovarian cancer cells (ATCC, Manassas, Va.), will be maintained in DMEM/F12 medium (Gibco, Invitrogen Canada, Inc., Burlington, ON, Canada) supplemented with 10% fetal bovine serum and 1% antibiotics-antimycotics (Sigma-Aldrich, St. Louis, Mo.). To prepare for injection, cells will be trypsinized and resuspended in serum-free medium at a concentration of 1×10⁷ cells/ml. Cell viability will be determined by trypan blue exclusion assay. Female Nu/Nu mice (Charles River Laboratories), 6 to 8 weeks old, will be inoculated with 10⁶ cells (100 μl) into the peritoneum. Tumors will be measured with calipers three times per week. When tumors reach approximately 1000 mm³ (i.e., in 1.5-2.5 weeks), the animals will be used for experiments. Two milliliters of fluorescently labeled 100 nm or 500 nm particles will be injected via tail vein in the mouse. Experiments will be performed with 2 different doses, 100 ug and 500 ug in 2 ml of sterile saline. 100 ug/2 ml is the minimal imageable dose to observe the particles in the vasculature while 500 ug/2 ml is the maximum dose injected into a mouse.

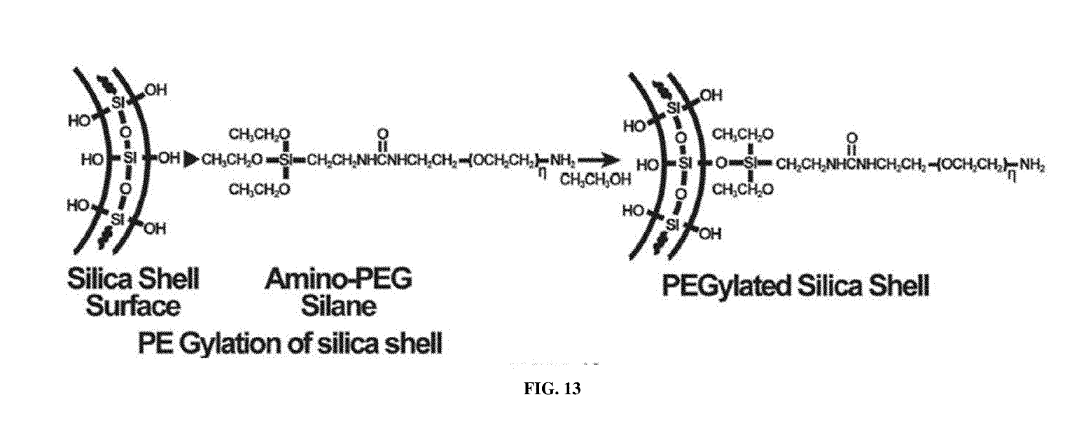

In order to extend the circulation lifetime of particles, particles of all sizes, targeted and untargeted will undergo PEGylation. PEGylation of nanoparticles can substantially increase the circulation of nanoparticles in vivo allowing particles to accumulate in the tumor bed and reduce immune response. PEGylation of the hollow silica particles is possible through well-known silane chemistry and commercially available PEG-Silane products through a plethora of manufacturers. A basic scheme of PEGylating the particles is shown in FIG. 13. Furthermore, amino-PEG-Silanes are also commercially available which conserves the NHS-linking chemistry and targeting potential of the particles to link NHS-Folate to the primary amine of the PEG as was previously done with 3-aminopropylsilane (FIG. 6). Carboxyl-PEG-Silane products are also available and would be used for non-targeted particles to ensure that non-targeted and targeted particles maintain similar (negative) surface charges.

For each particle size and dose, 5 mice with 5 tumors (totaling 20 mice with 20 tumors across all formulations) will be employed to obtain good statistical power. Animals will be imaged at 0, 1, 24, 48, and 72 hours after injection and sacrificed. The multiple time points will allow persistence in the tumor and circulation to be determined. At each imaging time point, sequences of ultrasound frames greater than 30 frames will be acquired in the tumor and the liver. At each location, the ultrasound transducer will be clamped in place to minimize motion artifacts. Ultrasound gel will be applied and the transducer will not be pressed hard against the tumor to affect the local blood circulation. Image sequences will be acquired through the center of the lesions by visually selecting the imaging planes with the largest tumor cross-section. The ultrasound imaging will be performed with a Siemens Sequoia scanner (with a GE Logiq E9 as a backup scanner) using contrast optimized imaging modalities. During the scans, maximum output power will be applied to achieve the highest particle signals as indicated with the preliminary data. After the post processing, the presence of the particles are highlighted, and they will be quantified by computing the mean brightness of the particles, which is separated from the tissue background by the post processing in selected regions of interest (ROI). The same post processing algorithm and parameters will be kept consistent for all datasets so that the particle signal in all samples may be compared quantitatively. The optimal dose and size of the particles will be determined by maximizing the mean brightness ratio between the tumors and the livers. These ratios will also be used as indicators of the binding of particles to the tumors. Depending on how well the particles perfuse through the tumors, partial volumes of the tumors may be selected as the ROIs. However, as-large-as-possible ROIs will be selected for the livers to form consistent baselines. After the animals are euthanized at 72 hrs, tumors and the livers will be fixed, sliced for histology. Fluorescence images and brightfield images of the histology slices will be acquired and compared with the ultrasound images (pre- and post-processing). The presence and distribution of the particles will be documented and confirmed with histology.

In another set of experiments a prostate cancer model will be used since it is also applicable to HIFU therapy and HIFU assisted drug therapy. LNCaP prostate cancer cells (ATCC, Manassas, Va.), will be maintained in RPMI medium (Gibco, Invitrogen Canada, Inc., Burlington, ON, Canada) supplemented with 10% fetal bovine serum and 1% antibiotics-antimycotics (Sigma-Aldrich, St. Louis, Mo.). To prepare for injection, cells will be trypsinized and resuspended in serum-free medium at a concentration of 1×10⁷ cells/ml. Cell viability will be determined by trypan blue exclusion assay. Male Nu/Nu mice (Charles River Laboratories), 6 to 8 weeks old, will be inoculated with 10⁶ cells (100 μl) into the peritoneum. Tumors will be measured with calipers three times per week. When tumors reach approximately 1000 mm³ (i.e., in 1.5-2.5 weeks), the animals will be used for experiments. Two milliliters of fluorescently labeled 100 nm or 500 nm particles will be injected via tail vein in the mouse. 2 different doses of 100 ug and 500 ug in 2 ml of sterile saline will be used. 100 ug/2 ml is the minimal imageable dose to observe the particles in the vasculature while 500 ug/2 ml is the maximum dose that has been safely injected into a mouse. For each particle size and dose, 5 mice with 5 tumors (totaling 20 mice with 20 tumors across all formulations) will be employed to obtain good statistical power. Animals will be imaged at 0, 1, 24, 48, and 72 hours after injection and sacrificed. During the scans, maximum output power will be applied to achieve the highest particle signals as indicated with the preliminary data. After the post processing, the presence of the particles are highlighted, and they will be quantified by computing the mean brightness of the particles, which is separated from the tissue background by the post processing in selected regions of interest (ROI). The optimal dose and size of the particles will be determined by maximizing the mean brightness ratio between the tumors and the livers. After the animals are euthanized at 72 hrs, tumors and the livers will be fixed, sliced for histology. Fluorescence images and brightfield images of the histology slices will be acquired and compared with the ultrasound images (pre- and post-processing). The presence and distribution of the particles will be documented and confirmed with histology.

Determine the Minimum Tumor Volume that is Imageable by Silica Nanoshells Via IV Injection.

Once the optimal particle size (100 nm vs 500 nm) and dose (100 μg/2 ml vs 500 μg/2 ml) of untargeted nanoshells is determined, targeting will be studied. The optimal particle size and dose will be tested untargeted vs folate targeted. The signal intensities from tumors will be compared. Female Nu/Nu mice (Charles River Laboratories), 6 to 8 weeks old, will be inoculated with 10⁶ cells (100 μl) intraperitoneally. Tumors will be measured with calipers three times per week. When tumors reach approximately 1 cm³ (i.e., in 1.5-2.5 weeks), the animals will be used for experiments. Two milliliters of fluorescently labeled targeted and untargeted particles will be injected via tail vein in the mice. The experiment will be repeated with 10 mice (10 tumors) for folate-targeted particles and 5 mice (5 tumors) for untargeted particles to supplement the 5 mice previously imaged. Animals will be imaged at 0, 1, 24, 48, and 72 hours after injection. The multiple time points will allow persistence in the tumor and circulation to be determined. At each imaging time point, sequences of ultrasound frames greater than 30 frames will be acquired in the tumor and, the liver. Performance of folate-targeted particles will be compared to untargeted particles by measuring the mean brightness ratio between the tumors and the livers. If it is determined that folate-targeted particles do not increase tumor enhancement over untargeted particles, αVβ3-targeted particles will be tested with the same experiments using an additional 10 mice (10 tumors).

In another experiment, once the optimal particle size (100 nm vs 500 nm) and dose (100 μg/2 ml vs 500 μg/2 ml) of untargeted nanoshells are determined, targeting will be studied. The optimal particle size and dose will be tested untargeted vs folate targeted. The signal intensities from tumors will be compared. Male Nu/Nu mice (Charles River Laboratories), 6 to 8 weeks old, will be inoculated with 10⁶ LNCaP prostate cancer cells (100 μl) intraperitoneally. Two milliliters of fluorescently labeled targeted and untargeted particles will be injected via tail vein in the mice. The experiment will be repeated with 10 mice (10 tumors) for folate-targeted particles and 5 mice (5 tumors) for untargeted particles to supplement the 5 mice previously imaged as described above. Animals will be imaged at 0, 1, 24, 48, and 72 hours after injection. At each imaging time point, sequences of ultrasound frames greater than 30 frames will be acquired in the tumor and, the liver. Performance of folate-targeted particles will be compared to untargeted particles by measuring the mean brightness ratio between the tumors and the livers. If it is determined that folate-targeted particles do not increase tumor enhancement over untargeted particles, αVβ3-targeted particles will be tested with the same experiments using an additional 10 mice (10 tumors).

After the animals are euthanized at 72 hrs, tumors and the livers will be fixed, sliced for histology. Fluorescence images and brightfield images of the histology slices will be acquired and compared with the ultrasound images (pre- and post-processing). The presence and distribution of the particles will be documented and confirmed with histology.

Determine the Minimum Tumor Volume that is Imageable by Silica Nanoshells Via IV Injection.

To determine the smallest tumor size that can be imaged by IV injection, the optimal particle type, targeting, injection dose, and imaging time point will be employed. A separate cohort of mice will be studied 3 days, 7 days and 14 days after cancer cell inoculations to examine the minimal tumor size that can be detected. Two orthogonal cross-section ultrasound images of the tumor will be taken to estimate the volume of the tumor, along with traditional caliper measurements. The entire tumor will be resected for histological analysis. The tumor volume derived from the histology will be used as the gold standard and compared to the tumor volume detected by imaging. The experiment will be repeated with 40 mice with 40 tumors.

Test Efficacy of Gas and Liquid Filled Nanoshells for HIFU—

Upon completion of the experiments above, the optimal nanoshells will be investigated for their potential as HIFU agents. Two modalities will be explored. First, using a single bolus injection of gas filled particles, it will then be determined if a tumor can be thermally ablated at a lower ultrasound power (mechanical index) compared to non-enhanced HIFU. Tumors will be grown in 10 mice. Half of the mice will be injected with a single bolus of optimal particles while the other half will be used as control; the dose size will be the largest studied in aim 2,500 μg/2 ml. After the particles are observed in the tumors, the particles will be subjected to high intensity ultrasound while the temperature of the tumor is monitored with MRI. Control experiments will be performed on tumor bearing mice using HIFU but with no silica shells for enhancement. The minimum mechanical index to raise the tumor temperature to 50 C will be determine for both the mice with and without the bolus injection of microshells. If the IV administration does not sufficiently lower the required mechanical index for raising the tumor temperature, the particles will be directly injected into the tumor. The experiments on human mastectomy tissue show that the particles are retained at the exact site of injection in tumor tissue.

In another experiment, a single bolus injection of PFC liquid/gas filled particles will be used to liquefy and ablate the bulk of the tumor and damage the surrounding vasculature feeding the tumor. Both purely mechanical/cavitation response via low duty cycle HIFU and including a thermal component with higher duty cycle HIFU will be investigated. Tumors will be grown in 30 mice divided into six groups varying either the HIFU duty cycle or the applied frequency. Each group of 5 mice will receive a single bolus injection of PFC liquid filled nanoshells, the nanoshells will be allowed to circulate within the mice for 24 hours prior to HIFU. Preliminary experiments have shown that HIFU with a duty cycle 2% at 1.1 MHz and 3 MPa while rapidly mechanically ablating tumor tissue is insufficient to cause thermal damage in the presence of nanoshells and will be used a starting point for experiments. The HIFU applied to the mice will be for 1 minute at 3 Mpa with duty cycles at 2%, 10%, and 50% with HIFU frequencies at either 1.1 or 3.3 MHz (3 duty cycles×2 frequencies=6 groups). With an additional 10 mice, control experiments will be performed on tumor bearing mice using HIFU but with no silica shells for enhancement. After HIFU, the tumor vasculature characteristics will be determined using conventional microbubbles (this technique is routinely used clinically to assess tumor status). After ultrasound analysis, the tumors will be resected and studied by histology. The efficacy of nanoshells and ratio of mechanical to thermal damage will be characterized by both US and histology analysis.

Second, using a bolus injection of liquid filled particles, one can determine if millimeter sized bubbled can be generated in the tumor vasculature to destroy the capillaries feeding the tumor. First, the tumors vasculature characteristics will be determined using conventional microbubbles (this technique is routinely used clinically to assess tumor status). Second, using a single bolus injection of liquid filled silica nanoparticles, it will be determined if the blood flow to the tumor can be disrupted by HIFU. Two tumors will be grown in 10 mice. Half of the mice will be injected with a single bolus of optimal liquid particles while the other half will be used as controls; the dose size will be the largest studied in aim 2,500 μg/2 ml. After the particles are observed in the tumors, the particles will be subjected to HIFU while the temperature of the tumor is monitored with MRI. Control experiments will be performed on tumor bearing mice using HIFU but with no silica shells for enhancement. Third, after the particles are stimulated into large 1 mm bubble (see image below), the vasculature of the tumor will be probed with CEUS will conventional microbubbles.

Cavitational Nanoshells and HIFU:

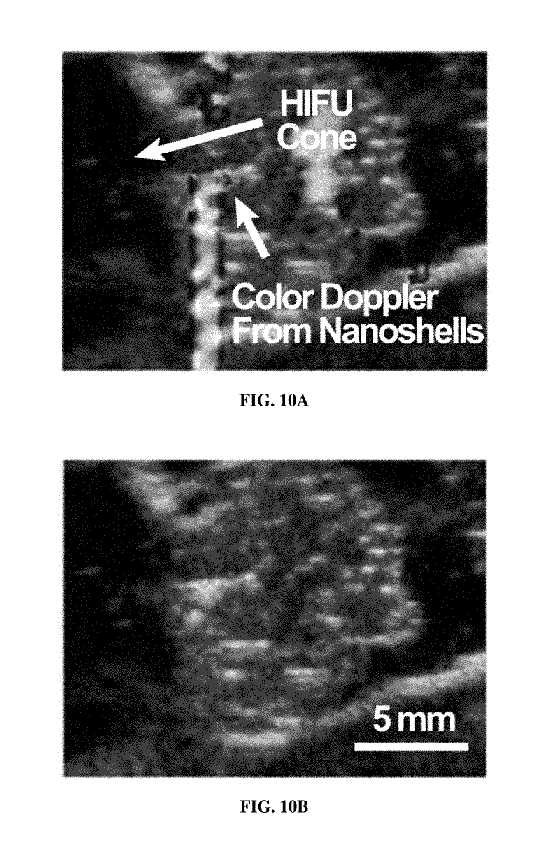

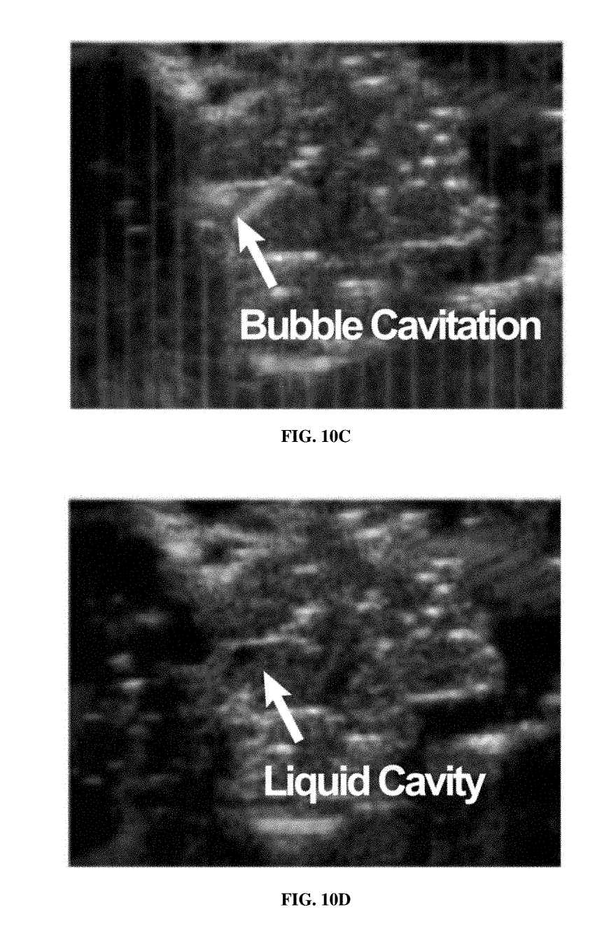

Nanoshells were tested ex vivo in excised human mastectomy tissue. For application in breast conservation surgery, the goal is to pre-operatively inject these particles via CT guidance in the same fashion that radioactive seeds or guide wires are currently implanted to help precisely localize the tumor for excision. Therefore, the particles remain stationary prior to and throughout surgical excision. As shown in FIG. 10A, the particles can be precisely injected next to a tumor margin and will not be transported away from site of localization (also confirmed by cross-sectional microscopy) thereby enabling multiple injections around the tumor to more thoroughly outline the margin. FIG. 10B contains a fluorescent microscopy image of a cross-sectional cut from the injection site. The fluorescence is from FITC that was covalently linked to the surface of the particle; the fluorescence being restricted to an area of several square millimeters is consistent with the volume that was injected initially, which reconfirms that the particles are localized at the injection site.

To demonstrate that nanoshells could potentially be used for cavitational HIFU therapy in humans, PFP liquid filled 500 nm nanoshells were injected intratumorally into excised mastectomy tissue and then HIFU was performed. As seen in FIG. 11A, the nanoshells are clearly visible under color Doppler ultrasound imaging. Once the HIFU is activated (FIG. 11C), the cavitation and bubble generation can clearly be seen at the sight where the color Doppler signal originated. Comparing the images between FIG. 11B before the HIFU was applied and FIG. 11D after the HIFU was applied, a cavity is clearly visible within the tumor. Using a vacuum or a syringe, it would be possible to drain the liquefied tissue from within the cavity and refill this pocket with a therapeutic or more nanoshells as described elsewhere herein.

As demonstrated above 2 μm nanoshells were shown to be able to detect intraperitoneal (IP) late stage tumors by ultrasound. By using gamma scinitigraphy and color Doppler ultrasound it was demonstrated that 500 nm nanoshells are well retained by tumors when administered intratumorally. To further demonstrate that nanoshells can be used to readily detect and image multiple tumors as well as accumulate in tumors for HIFU therapy, nanoshells were labeled with radioactive 111-indium-DTPA (diethylenetriamine pentaacetate) and injected into Py8119 breast tumor bearing mice. Each mouse was implanted with 2 tumors, one on each of its flanks. The DTPA was covalently anchored to the nanoshell surface and is a well-known chelator of indium and is commonly used to study biodistribution of various nano-formulations. Each mouse was injected via tail vein with 100 μl of nanoshells at 4 mg/ml with 15-20 μCi/dose and planar γ-scintigraphic imaging was performed as shown in FIGS. 8A-8D. After 72 hours, animals were sacrificed, the organs were harvested, and total organ radioactivity was measured with the use of a γ-counter. FIG. 8A shows that the particles are initially spread throughout the entire body of the animal with an initial high accumulation in the liver. However, even immediately after initial injection of the nanoshells seen in FIG. 8A, an outline of the tumors (two bilateral lobes on the flanks near the bottom of the mouse at the center of the image) can be observed at the bottom of the mouse. The tumor images become increasingly distinct over time in FIG. 8C-D. It is hypothesized that the nanoparticles retained by the tumor are a product of the enhanced permeation and retention (EPR) effect which has been documented for various in vivo tumor models. The poorly developed vasculature in the tumor sequesters and retains macromolecules and nanoparticles within the tumor due to poor circulation, drainage and leakiness. Furthermore, the amount of nanoshells retained by each tumor were approximately constant, when normalized by tumor mass, as evidenced by the nearly equal values of percent injected dose per gram tumor (FIG. 8E).

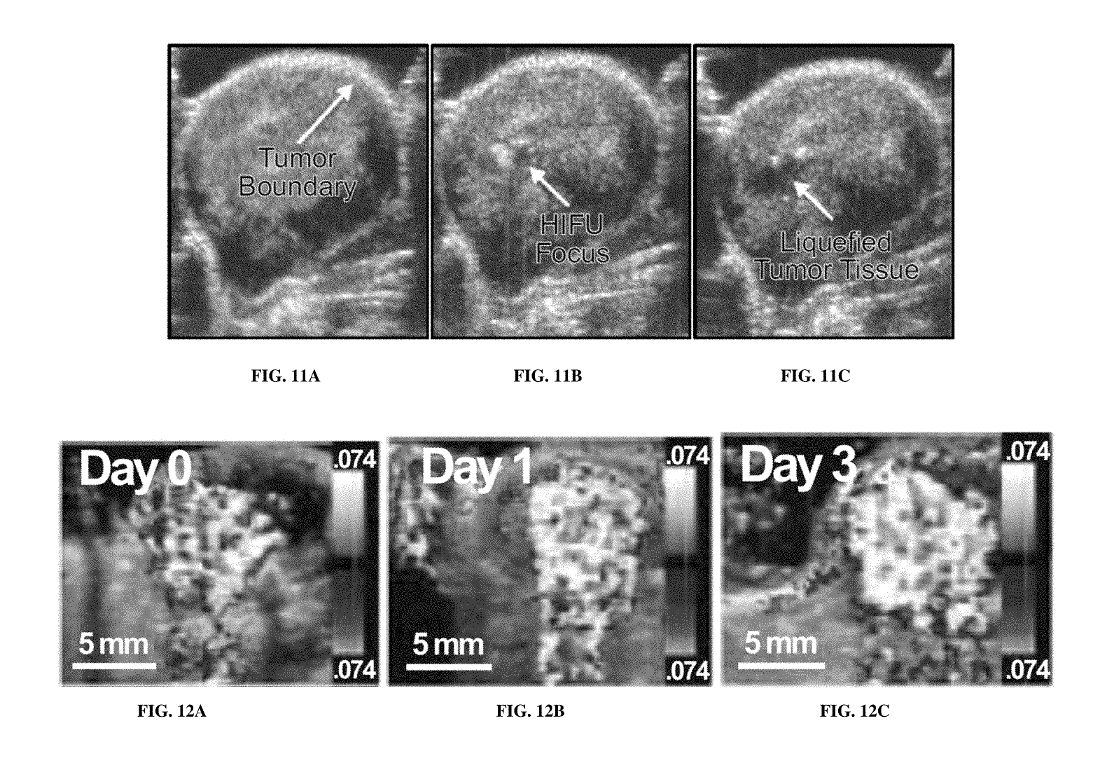

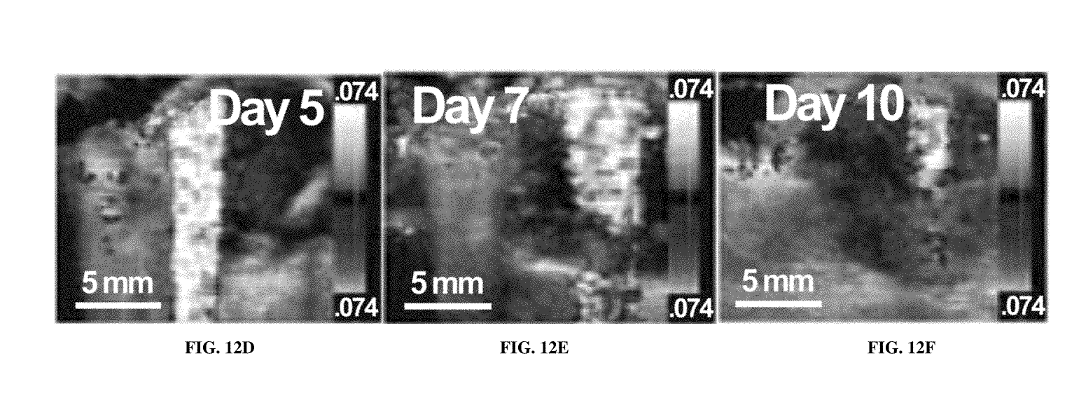

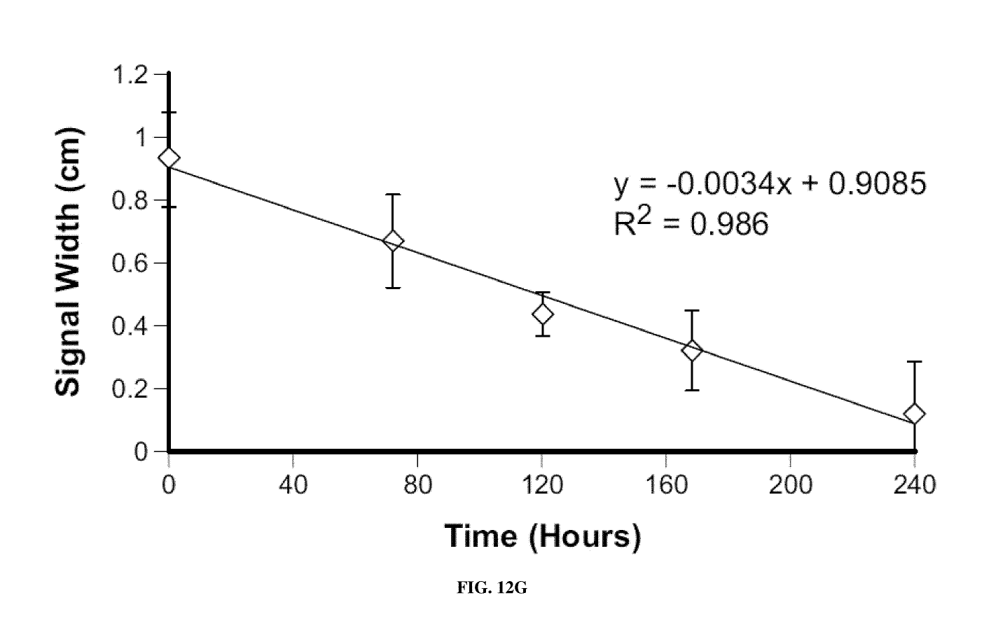

Once it was determined that nanoshells can accumulate in this tumor model, nanoshells were administered intravenously into the same Py8119 breast tumor bearing Nu/Nu mice. PFP Liquid filled nanoshells were allowed to circulate and accumulate in the tumors for 24 hours prior to HIFU administration. HIFU was applied for 1 minute at 3 MPa and 1.1 MHz with a 2% duty cycle. As HIFU is applied, the nanoshells are fractured and the liquid perfluoropentane within the nanoshells undergoes acoustic droplet vaporization (FIG. 12B) and then begins to cavitate. This cavitation is powerful enough that it liquefies the tissue within the focal volume (FIG. 12C) of the HIFU and tissue that is outside of the focal volume remains unaffected. FIG. 11 demonstrates that perfluoropentane filled 500 nm Fe—SiO₂ nanoshells are capable of being used as HIFU sensitizing agents, specifically for enhancing mechanical cavitation and liquification of tissue in vivo.

Longterm Imaging Marker:

It has been previously shown that PFC gas filled nanoshells could be used for extended ultrasound imaging. Fe-doped nanoshells were injected intratumorally into eight Py8119 breast tumor bearing Nu/Nu mice. The particles were imaged by color Doppler ultrasound over the course of 10 days (FIG. 13A-F). The color Doppler signal width was measured and plotted against time (FIG. 13G). It was found that the signal persisted for ten days and decayed linearly with imaging over time. To maximize the number of simultaneous nanoshells being imaged, a higher mechanical index (1.9) (ultrasound power) was applied. As a result of using a higher mechanical index, a substantial degree of shadowing was observed from the high reflectivity of the particles. This shadowing was observed under ultrasound as an exaggerated color Doppler tail, which embellishes the color Doppler signal in the Y-axis (FIG. 13D). To overcome interference from the observed shadowing, the signal width was used to measure the signal decay instead of the signal area.

However, after 10 days no signal could be observed. In order to retain signal for a longer period of time in vivo it would be necessary to prevent the perfluorocarbon within the nanoshell from escaping and prevent anything within body fluids from penetrating the nanoshells. The proposed method to achieve this would be to fluorinate the surface of the nanoshell which would create a “non-wetting” surface. This is done by suspending the nanoshells in a perfluorocarbon solution and then adding an excess of a flourous alkoxysilane (e.g., trialkoxysilane such as, perfluorooctyltriethoxysilane). The more fluorinated the alkoxysilane is the more soluble it will be in the PFC liquid and the less likely it would be that PFC from within the nanoshell would escape. This solution is then de-gassed in a bath sonicator to remove any gasses within the nanoshells allow for the nanoshells to be filled with and trap the perfluorocarbon liquid, followed by mixing on a vortex. The silane reaction and the flourous phase effectively close up the pores throughout the nanoshell and dramatically reduce the interaction of the nanoshells with the local environment. This may make for nanoshells which could have an indefinite in vivo lifetime which could still be imaged by ultrasound imaging modalities.

Nanoshell HIFU Enhanced Local (IT) Drug Delivery.

HIFU will be employed to ablate the tumor after IV injection of nanoshells. To remove the remaining cancer cells a nanoformulation (Doxil) will be injected into the HIFU pocket. It has been previously observed that hyperthermic therapies can increase the ability of tumors to retain drugs as well as nanoformulations. Doxil is used since it is FDA approved, however other therapeutics can be substituted for Doxil. Nanoformulations are known to have typically poor tissue penetration as a result of relatively large size with respect to the functional porosity of tissue. As a result many nanoformulations are unable to effectively deliver drugs uniformly throughout tumors. Furthermore injecting therapeutics intratumorally frequently proves ineffective due to high intratumoral pressures as well as rapid diffusion away from the tumor. By creating a pocket within the center of a tumor, the center of the tumor can effectively become a drug depot and the poor diffusion properties which previously prevented the particles from penetrating the tumors are now advantageous in preventing their escape. There has been evidence that cavitational HIFU can enhance macromolecule or drug retention within tumors. 30 tumor bearing animals in three groups (10 animals per group: HIFU/Doxil, Doxil alone, and HIFU alone) will be used to investigate the efficiency of the HIFU combined with the Doxil. LNCaP tumors will be grown IP as previously described. Animals will receive a dose of nanoshells based on the findings of previous aims. Nanoshells will be allowed to circulate for 24 hours prior to insonation; HIFU will be applied for 1 min, at the optimal parameters determined in Aim 3. The liquid will be removed from the region of the tumor which underwent mechanical cavitation and the cavity will be filled with a dose of Doxil reflective of a standard full dose per each animals mass. For HIFU alone, the cavity will be filled with saline. Doxil alone will be delivered via intratumoral injection. Mice will receive a treatment of nanoshells/HIFU/therapeutic on a weekly basis for 10 weeks. Disease progression will be monitored by measuring tumor size daily with calipers and weekly by diagnostic ultrasound. After 10 weeks, animals will be sacrificed; tumors and organs will be analyzed by histology.

Nanoshell HIFU Enhanced Oncolytic Local (IT) Viral Therapy.

Oncolytic viruses are known to be highly effective when injected locally but there are challenges to injecting a sufficient dose due to high pressure and density within many tumors. Cavitational HIFU will be employed to create a pocket inside the tumor after IV injection. To remove the remaining cancer cells an Oncolytic virus will be injected into the cavity. UCSD has developed several effective Oncolytic viruses, and a proprietary technology enables transfection of cells which do not even express the appropriate receptor. This may enable effective tumor treatment even with local metastasis because once the virus transfects the cells, it will continue to replicate and further transfect cells. The cavitational HIFU may be optimal since it leaves viable cells for transfection. 30 tumor bearing animals in three groups (10 animals per group: HIFU/Liposomal encapsulated Oncolytic viruses, HIFU/Oncolytic viruses and Oncolytic viruses alone will be used to investigate the efficiency of the HIFU combined with the oncolytic viruses. LNCaP tumors will be grown IP as previously described in Aim 1. Animals will receive a dose of nanoshells based on the findings of previous aims. Nanoshells will be allowed to circulate for 24 hours prior to insonation; HIFU will be applied for 1 min, at 1.1 MHz at 3 MPa at a low duty cycle to create an intratumoral cavity. The liquid will be removed with a vacuum line and the cavity will be filled with a dose of encapsulated oncolytic viruses or oncolytic viruses reflective of a standard full dose per each animals mass (˜5×10⁹ pfu/mouse). Oncolytic virus alone will be delivered via intratumoral injection. Mice will receive a treatment of nanoshells/HIFU/therapeutic on a weekly basis for 10 weeks. Disease progression will be monitored by measuring tumor size daily with calipers and weekly by diagnostic ultrasound. After 10 weeks, animals will be sacrificed; tumors and organs will be analyzed by histology.

A number of embodiments have been described herein. Nevertheless, it will be understood that various modifications may be made without departing from the spirit and scope of this disclosure. Accordingly, other embodiments are within the scope of the following claims.

What is claimed is:

1. A method of imaging tissue, comprising:
administering hollow silica nanoshells to a subject, the hollow silica nanoshells structured to include a porous shell comprising polymeric R-group reinforced silica and having a hollowed interior, wherein the hollow silica nanoshells are operable to concentrate at a tissue located in a region of the subject's body after circulating through the subject's body after administering;
directing ultrasonic energy at the region of the subject's body to cause the hollow silica nanoshells to produce a detectable signal at the tissue; and
imaging the tissue based on the detectable signal using an ultrasound imaging technique.

2. The method of claim 1, further comprising:
prior to administering, dying the hollow silica nanoshells with an ink dye.

3. The method of claim 2, wherein dying the hollow silica nanoshells with the ink dye comprises India ink.

4. The method of claim 1, wherein the polymeric R-group reinforced silica includes a formula: 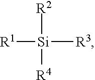
wherein R¹-R⁴ are independently selected from the group consisting of H, D, optionally substituted (C₁-C₁₈)alkyl, optionally substituted (C₁-C₁₈)alkenyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether;
wherein at least one of R¹-R⁴ is an optionally substituted alkoxy, and
wherein, when three of R¹-R⁴ are methoxy groups, the fourth R group is selected from the group consisting of D, optionally substituted (C₁-C₁₈)alkyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted (C₂-C₁₈)alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether.

5. The method of claim 1, wherein the hollow silica nanoshells are fabricated by:
forming a polymer template core particle by mixing polystyrene or latex beads with a polyamine, polyamino acids, cationic polymers, or molecular anchors with a cationic headgroup in an aqueous solution;
forming an outer layer on the polymer template core particle by adding a mono-, di-, tri- or tetra-alkoxysilane to the aqueous solution to bind to the surface of the polymer template;
calcining the polymer template core particle to render a silica shell from the outer layer and polymeric R groups from the polymer template core particle to produce the hollow silica nanoshells, wherein the calcining removes the polystyrene or latex beads; and
drying the hollow silica nanoshells.

6. The method of claim 1, wherein the hollow silica nanoshells are PEGylated or folate functionalized.

7. The method of claim 1, wherein the porous shell of the hollow silica nanoshells includes one or more fluorinated alkyl groups, and the hollow silica nanoshells are hydrophobic.

8. The method of claim 1, wherein the hollow silica nanoshells include a diameter between 1 nm to 100 nm.

9. The method of claim 1, wherein the hollow silica nanoshells include a surface area between 100 m²/gram to 1000 m²/gram.

10. The method of claim 1, wherein the tissue includes lung tissue, breast tissue, liver tissue, or tissue of the head and neck including squamous cells.

11. The method of claim 1, wherein the tissue includes cancer, a tumor or neoplasm.

12. The method of claim 1, wherein imaging the tissue using the ultrasound imaging technique includes imaging the hollow silica nanoshells in the tissue with contrast pulse sequencing ultrasound imaging.

13. The method of claim 1, wherein imaging the tissue using the ultrasound imaging technique includes imaging the hollow silica nanoshells in the tissue with harmonic ultrasound imaging.

14. The method of claim 1, wherein imaging the tissue using the ultrasound imaging technique includes imaging the hollow silica nanoshells in the tissue with color Doppler ultrasound imaging.

15. The method of claim 1, wherein administering the hollow silica nanoshells to the subject includes preoperatively injecting the hollow silica nanoshells into the tissue under CT guidance, and wherein the CT-guided hollow silica nanoshells-injected tissue includes a tumor margin under CT guidance.

16. A method of imaging cancer, tumors or neoplasms, comprising
administering hollow silica nanoshells to a subject having a cancer, a tumor, or a neoplasm; and
directing ultrasonic energy to the subject to cause the hollow silica nanoshells to produce a detectable signal, wherein the hollow silica nanoshells concentrate at a site of the cancer, tumor, or neoplasm,
wherein the hollow silica nanoshells are formed from a structure comprising:
a degradable nanotemplate comprising a polyamine or polycarboxylic acid functionalized surface layer, and
a layer of a compound comprising the structure of Formula I: 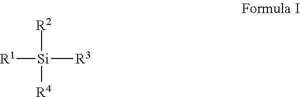
wherein R¹-R⁴ are independently selected from the group consisting of H, D, optionally substituted (C₁-C₁₈)alkyl, optionally substituted (C₁-C₁₈)alkenyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether;
wherein at least one of R¹-R⁴ is an optionally substituted alkoxy, and
wherein, when three of R¹-R⁴ are methoxy groups, the fourth R group is selected from the group consisting of D, optionally substituted (C₁-C₁₈)alkyl, optionally substituted (C₁-C₁₈)alkynyl, optionally substituted (C₁-C₁₈)cycloalkyl, optionally substituted (C₁-C₁₈)cycloalkenyl, optionally substituted heterocycle, optionally substituted aryl, optionally substituted mixed ring system, optionally substituted (C₂-C₁₈)alkoxy, halo, hydroxyl, carbonyl, aldehyde, haloformyl, carboxylate, carboxyl, ester, ether, amino, carboxamide, ketimine, aldimine, imide, azo, azide, cyanate, isocyanate, nitrate, nitrile, isonitrile, nitro, nitroso, thiol, sulfide, sulfinyl, sulfonyl, sulfino, sulfo, thiocyanate, isothiocyanate, carbonothioyl, phosphine, phosphono, phosphate, boronate, borono, borino, and silyl ether.

17. The method of claim 16, wherein the degradable nanotemplate is at least partially degradable, and wherein the hollow silica nanoshells include nondegraded portions of the nanotemplate.

18. The method of claim 16, wherein the hollow silica nanoshells include a diameter between 10 nm to 3000 nm.

19. The method of claim 16, wherein the hollow silica nanoshells include an ink dye.

20. The method of claim 16, further comprising:
imaging hollow silica nanoshells in or proximate to the cancer, tumor or neoplasm using an ultrasound imaging technique including contrast pulse sequencing ultrasound imaging, harmonic ultrasound imaging, or color Doppler ultrasound imaging.
